(19) United States
(12) Patent Application Publication
(10) Pub. No.: US 2026/0159818 A1
(43) Pub. Date: Jun. 11, 2026

(54) OXIDATION OF STEROIDS

(71) Applicant: Oxford University Innovation Limited, Oxford (GB)

(72) Inventors: Luet Lok WONG, Oxford (Oxfordshire) (GB); Wenyu CHEN, Oxford (Oxfordshire) (GB)

(21) Appl. No.: 19/104,130

(22) PCT Filed: Aug. 16, 2023
(86) PCT No.: PCT/GB2023/052151

§ 371 Date: Feb. 14, 2025

(30) Foreign Application Priority Data
Aug. 16, 2022 (GB) 2211941.6

(51) Int. Cl. C12N 9/02; C07C 35/21; C07D 303/14; C12N 15/52; C12P 7/02; C12P 33/00

(52) U.S. Cl. CPC C12N 9/0071; C07C 35/21; C07D 303/14; C12N 9/0042; C12N 15/52; C12P 7/02; C12P 33/00; C12Y 106/02004; C12Y 114/14001

(57) ABSTRACT

The present invention relates to processes for the oxidation of secosteroids using mutant Cytochrome P450 family 102A sub-family member (CYP102A) enzymes. The invention also relates to mutant CYP102A enzymes, polynucleotides encoding the enzymes, cells expressing the enzymes, transgenic animals or plants comprising the enzymes, and compounds produced by the processes.

### FIELD OF THE INVENTION

The disclosure relates to processes for the oxidation of steroids and polypeptides for use in the process.

### BACKGROUND OF INVENTION

Vitamin D (calcitriol) is a hormone which binds to nuclear receptors that activate and regulate the expression of numerous genes. Amongst its many physiological roles, vitamin D is crucial in the maintenance of blood calcium levels. Although its effect is mainly associated with bone effects via regulation of calcium homeostasis, vitamin D has also been linked to biological functions such as the immune system, potassium homeostasis and anti-cancer regulatory properties. It is approved for use as a general medicine. On the other hand, its low natural abundance and the lack of viable synthetic routes mean that the more readily available precursor vitamin D₃ is used as a supplement/precursor in most applications. Vitamin D₃ is converted to 25-(OH)-D₃, the circulating form of vitamin D, in the liver by the cytochrome P450 enzyme CYP27A1 (FIG. 1). 25-(OH)-D₃ is then converted to 1,25-dihydroxy-vitamin D₃ (calcitriol), the active form of vitamin D, in the kidneys by the enzyme CYP27B1.

25-(OH)-D₃ is a reliable indicator of vitamin D₃ levels in the blood. Maintaining recommended levels of this in serum (30-60 ng/mL) has been shown to be vital for maintaining the functions associated with vitamin D and general health.

There are two main sources of vitamin D₃: UV light and food. However, each source on their own would not normally be sufficient. UV light acts on 7-dehydrocholesterol under the skin to form pre-vitamin D₃ which isomerises thermally to vitamin D₃. This process relies on exposure to sunlight and is seasonally and geographically dependent. Except for fatty fish, few foods contain significant amounts of vitamin D₃. As a result, food fortification with vitamin D precursors has gained significant interest.

Human vitamin D supplements in the form of vitamin D₃ are routinely available. It is recommended for some section of the population, especially over the winter months when there is less exposure to sunlight and is used in clinical settings for patients with compromised immune and organ functions. Another major application is in animal feed, to boost weight, health and yield in husbandry. However, the low solubility of vitamin D₃ in gastrointestinal fluid and low bioavailability reduces the efficacy of orally administered vitamin D₃ supplements.

It is recognised that the circulating form of vitamin D, 25-(OH)-D₃, would be more effective in most applications. Indeed, 25-(OH)-D₃ is an approved medication, and 25-(OH)-D₃ is a more effective animal feed than vitamin D₃. Moreover, a link to cancer prevention has been established for 25-(OH)-D₃. The potential use of nano-encapsulation of active vitamin D₃ metabolites such as 25-(OH)-D₃ in chemotherapy has been suggested. Recently, it has been reported that a correlation between vitamin D deficient patients and more severe cases of Covid-19 may exist. Vitamin D₃ supplementation is proposed as an additional therapeutic strategy due to its anti-inflammatory and immune system regulatory functions. Other studies which show a link between Covid-19 severity and phosphate levels in the blood, further support the hypothesis that vitamin D₃, a phosphate metabolism regulator, can play a role in the progression of this disease.

Despite strong evidence for the higher efficacy of 25-(OH)-D₃ in multiple applications, its wide adaptation is limited by difficulties in production. Chemical processes for its synthesis from vitamin D₃ suffer from low yields due to the need to oxidise an unactivated aliphatic C—H bond at the C25 position. This step is difficult to achieve catalytically by chemical systems. Current industrial production of 25-(OH)-D₃ starts from 5,7,24-cholestatrienol, which is produced by fermentation using a mutant strain of Saccharomyces cerevisiae. The trienol is chemically oxidised to the 24,25-epoxide, reduced to the 25-alcohol which is converted photochemically to 25-(OH)-D₃ as well as other products. 25-(OH)-D₃ is recovered by crystallisation.

Vitamin D₂ (ergocalciferol or calciferol) and vitamin D₃ differ by the presence of the C22-C23 double bond and the C28 methyl group in vitamin D₂ (FIG. 2). Vitamin D₂ is formed by UV irradiation of ergosterol and is found in some plants and in mushrooms whereas vitamin D₃ is isolated from animals. The rigidity of the double bond and altered steric demand introduced by the methyl group can affect P450 substrate recognition, as demonstrated by the oxidation of vitamin D₂ to its circulating form, 25-hydroxy-vitamin D₂, by a different liver P450 enzyme (CYP2R1) from that for vitamin D₃ (CYP27A1). Likewise, CYP2R1 does not oxidise vitamin D₃. However, both 25-hydroxy vitamin-D₂ and -D₃ are oxidised by CYP27B1 in the kidneys to their respective active forms. The active form of vitamin D₂, 1,25-dihydroxy-vitamin D₂ (ercalcitriol), has similar binding affinity to the vitamin D receptor as calcitriol, the active form of vitamin D₃.

The P450 enzymes CYP27A1 and CYP21B1 catalyse the physiological sequential C25 then Cl oxidation of vitamin D₃ to the active form of vitamin D. The liver enzyme CYP2R1 is responsible for the C25-hydroxylation of vitamin D₂ to 25-(OH)-Vitamin D₂; CYP27A1 does not catalyse this reaction. These membrane-bound enzymes have low stability and activity. They are difficult to express in heterologous organisms, which make them unsuitable for large scale production.

Many microorganisms have been screened for vitamin D₃ oxidation, and isolation of new strains with such activity continues today. The activity and conversion have been reviewed in recent reports on strain isolation and characterisation (Schmitz, et al., Chembiochem 2021, 22, 2266-2274; Tang et al., Appl. Microbiol. Biotechnol. 2020, 104, 765-774). The earliest reports were of Streptomyces strains that oxidised vitamin D₃ at C1 and C25 to form human metabolites (Sasaki, et al., Appl. Environ. Microbiol. 1991, 57, 2841-2846). Amycolata autotrophica FERM BP-1573 transformed vitamin D₃ to 25-(OH)-D₃ (Sasaki et al., Appl. Environ. Microbiol. 1992, 38, 152-157; Takeda et al., J. Ferment. Bioeng. 1994, 78, 380-382). Pseudonorcadia autotrophica ID9302 also performed this reaction (Kang et al., Biotechnol. Bioproc. Eng. 2006, 11, 408-413), initially at low yield but up to 356 mg/L after screening and selection of mutants generated by UV irradiation and optimisation of process parameters, corresponding to a space-time yield (STY) of 2.97 mg/L/h (Kang et al., Biotechnol. Lett. 2015, 37, 1985-1904). A more recent isolate, Pseudonorcadia autotrophica CGMCC5098, showed a high yield of 25-(OH)-D₃ of 639 mg/L from a 120-hour fermentation, i.e., a STY of 5.33 mg/L/h (Luo et al., Biocatal. Biotransform. 2017, 35, 11-18). A pathogenic Bacillus cereus strain zju 4-2 was reported to produce up to 830 mg/L of 25-(OH)-D₃ from vitamin D₃ in a STY of 25.9 mg/L/h (Tang et al., Appl. Microbiol Biotechnol. 2020, 104, 765-774). A new bacterial isolate, Kutzneria albida, oxidised both vitamins D₂ and D₃ to the 25-(OH) derivative with yields of 13.7 mg/L and 70.4 mg/L respectively at a maximal STY of 1.47 mg/L/h (Schmitz et al., Chembiochem 2021, 22, 2266-2274). None of these absolute mass yields or STYs are sufficient for industrial production.

CYP105A1 from Streptomyces griseolus showed 55% amino acid sequence identity to CYP105A2 responsible for vitamin D₃ oxidation by Amycolata autotrophica. The recombinant form was engineered to increase the C25 oxidation activity of both vitamins D₂ and D₃, although the productivity and yields are low (Hayashi et al., Biochemistry, 2008, 47, 11964-11972; Yasutake et al., J. Biol. Chem. 2010, 285, 31193-31201; Yasuda et al., Biochem. Biophys. Res. Commun. 2017, 486, 336-341). Abdulmughni et al. characterised CYP109A2 from Bacillus megaterium as a vitamin D₃ hydroxylase (Abdulmughni et al., FEBS J. 2017, 284, 3881-3894; J. Biotechnol. 2017, 243, 38-47) and expressed an engineered variant in Bacillus megaterium to produce 282.7 mg/L of 25-(OH)-D₃ with a STY of 5.90 mg/L/h (Abdulmughni et al., J. Biotechnol. 2021, 325, 355-359). Pseudonocardia autotrophica CYP107BR1 (Vdh) has been engineered to increase its vitamin D₃ C25 oxidation activity, and expression of this variant in Rhodococcus erythropolis accompanied by membrane permeabilisation using nisin increased 25-(OH)-D₃ production to 573 mg/L (Yasutake et al., Chembiochem 2013, 14, 2284-2291). However, the product concentrations, reaction rates and STYs of these systems are not sufficiently high for economically viable industrial production of 25-(OH)-D₂ and -D₃.

There is a need for new methods to catalyse the selective oxidation of vitamin D₂ and D₃ to 25-(OH)-D₂ and 25-(OH)-D₃, respectively.

### SUMMARY OF INVENTION

The inventors have surprisingly discovered that a CYP102A enzyme may be mutated to enable selective oxidation of secosteroid substrates. The mutant CYP102A enzymes are able to catalyse a reaction with high product selectivity (i.e. few, if any, undesired products are produced), high conversion rate (the percentage of substrate converted to product) and with a high turnover number. In contrast, the wild-type enzyme does not exhibit any detectable oxidation of secosteroid substrates.

Thus, the inventors have provided a significant contribution to the art by providing an efficient method for producing 25-hydroxy-vitamin D₃ [25-(OH)-D₃, calcifediol or calcidiol] by direct oxidation of vitamin D₃ (cholecalciferol or colecalciferol), and for producing 25-hydroxy-vitamin D₂ [25-(OH)-D₂] by direct oxidation of vitamin D₂ (ergocalciferol, calciferol), using engineered variants of cytochrome P450_{BM3}.

Accordingly, the invention provides a process for oxidising a secosteroid, comprising the step of contacting said secosteroid with a mutant CYP102A (Cytochrome P450 family 102A sub-family member) enzyme, wherein said CYP102A enzyme comprises a heme monooxygenase domain comprising a P450 fold, and said mutant CYP102A enzyme comprises substitution(s) in the polypeptide chain of a wild-type CYP102A enzyme at one or more positions, thereby enhancing monooxygenase activity and/or altering product selectivity of the mutant enzyme.

The invention also provides a mutant CYP102A (Cytochrome P450 family 102A sub-family member) enzyme, wherein said CYP102A enzyme comprises a heme monooxygenase domain comprising a P450 fold, and said mutant CYP102A enzyme comprises substitution(s) in the polypeptide chain of a wild-type CYP102A enzyme at one or more positions corresponding to amino acid residue positions 435, 82, 184, 260 and/or 72 of SEQ ID NO: 2, thereby enhancing monooxygenase activity and/or altering product selectivity of the mutant enzyme.

The invention also provides a polynucleotide which comprises a sequence which encodes an enzyme of the invention or a cell which expresses an enzyme of the invention. The invention also provides a transgenic animal or plant comprising cells of the invention.

The invention also provides the compounds (1S,Z)-3-(2-((1R,3aS,7aR,E)-7a-methyl-1-((2R)-5-(2-methyloxiran-2-yl) pentan-2-yl) octahydro-4H-inden-4-ylidene)ethylidene)-4-methylenecyclohexan-1-ol (i.e. 25,26-epoxy-vitamin D₃), and (3S,6R,E)-6-((1R,3aS,7aR,E)-4-((Z)-2-((S)-5-hydroxy-2-methylenecyclohexylidene)ethylidene)-7a-methyloctahydro-1H-inden-1-yl)-2-methylhept-4-ene-2,3-diol (i.e. 25,28-dihydroxy-vitamin D₂).

### BRIEF DESCRIPTION OF THE FIGURES

FIG. 1—The biosynthesis of vitamin D (calcitriol) by sequential oxidation of vitamin D₃ by P450 enzymes.

FIG. 2—The biosynthesis of ercalcitriol by sequential oxidation of vitamin D₂ by P450 enzymes.

FIG. 3—The hydroxylation of cholecalciferol (vitamin D₃) catalysed by selected cytochrome CYP102A1 variants.

FIG. 4—The hydroxylation of calciferol (vitamin D₂) with second generation CYP102A1 variants.

### BRIEF DESCRIPTION OF SEQUENCE LISTING

SEQ ID NO: 1-CYP102A1 nucleotide sequence

SEQ ID NO:2-CYP102A1 amino acid sequence

SEQ ID NO: 3 (CYP102A1, residues 1-470).

SEQ ID NO: 4 (CYP102A2, P450 domain corresponding to 1-470 of CYP102A1).

SEQ ID NO: 5 (CYP102A3, P450 domain corresponding to 1-470 of CYP102A1).

SEQ ID NO: 6 (CYP102A4, P450 domain corresponding to 1-470 of CYP102A1).

SEQ ID NO: 7 (CYP102A5, P450 domain corresponding to 1-470 of CYP102A1).

SEQ ID NO: 8 (CYP102A6, P450 domain corresponding to 1-470 of CYP102A1).

SEQ ID NO: 9 (CYP102A7, P450 domain corresponding to 1-470 of CYP102A1).

SEQ ID NO: 10 (CYP102A8, P450 domain corresponding to 1-470 of CYP102A1).

SEQ ID NO: 11 (CYP102A9, P450 domain corresponding to 1-470 of CYP102A1).

SEQ ID NO: 12 (CYP102A10, P450 domain corresponding to 1-470 of CYP102A1).

SEQ ID NO: 13 (CYP102A11, P450 domain corresponding to 1-470 of CYP102A1).

SEQ ID NO: 14 (CYP102A12, P450 domain corresponding to 1-470 of CYP102A1).

SEQ ID NO: 15 (CYP102A13, P450 domain corresponding to 1-470 of CYP102A1).

SEQ ID NO: 16 (CYP102A14, P450 domain corresponding to 1-470 of CYP102A1).

SEQ ID NO: 17 (CYP102A15, P450 domain corresponding to 1-470 of CYP102A1).

SEQ ID NO: 18 (CYP102A16, P450 domain corresponding to 1-470 of CYP102A1).

SEQ ID NO: 19 (CYP102A25, P450 domain corresponding to 1-470 of CYP102A1).

SEQ ID NO: 20 (CYP102A26, P450 domain corresponding to 1-470 of CYP102A1).

SEQ ID NO: 21 (Krac9955 (A.K.A. CYP102A18), P450 domain corresponding to 1-470 of CYP102A1).

SEQ ID NO: 22 (Krac0936 (A.K.A. CYP102A18), P450 domain corresponding to 1-470 of CYP102A1).

SEQ ID NO: 23 (CYP102B1, P450 domain corresponding to 1-470 of CYP102A1).

### DETAILED DESCRIPTION

The invention relates to a process for oxidising a secosteroid. The process comprises the step of contacting said secosteroid with a mutant CYP102A (Cytochrome P450 family 102A sub-family member) enzyme, wherein said CYP102A enzyme comprises a heme monooxygenase domain comprising a P450 fold, and said mutant CYP102A enzyme comprises substitution(s) in the polypeptide chain of a wild-type CYP102A enzyme at one or more positions, thereby enhancing monooxygenase activity and/or altering product selectivity of the mutant enzyme.

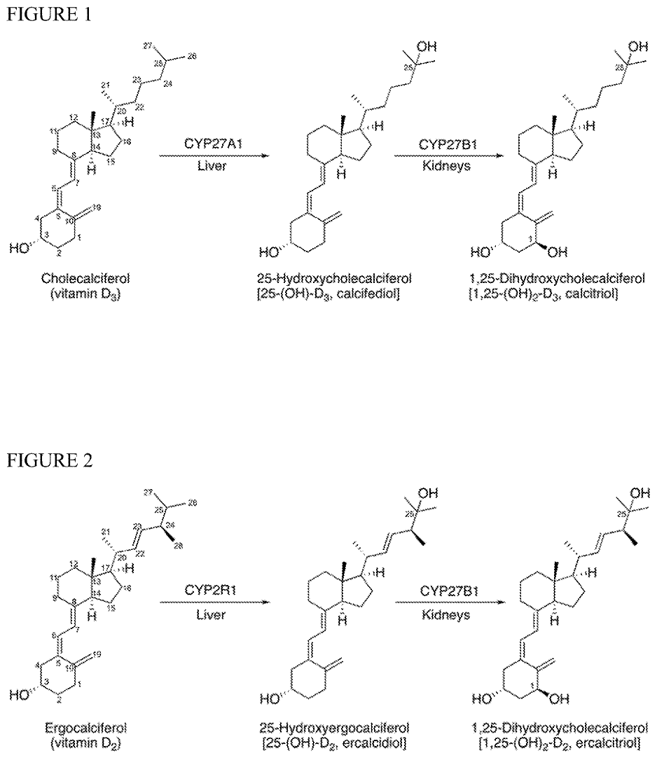

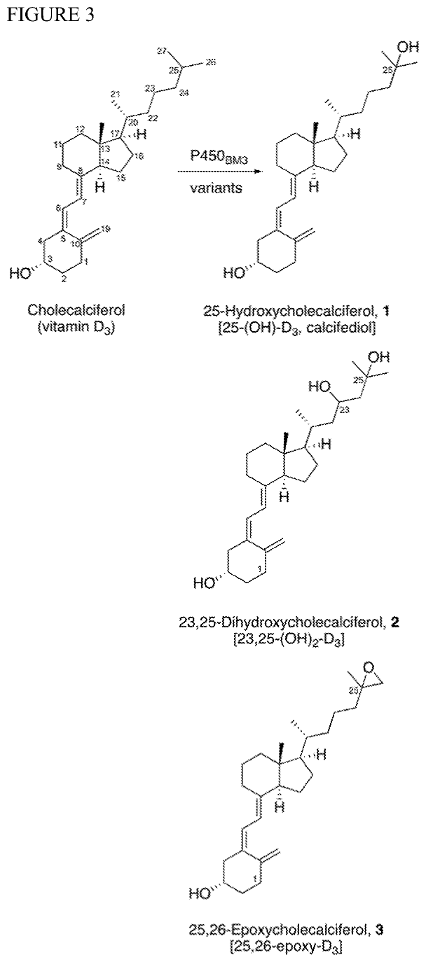

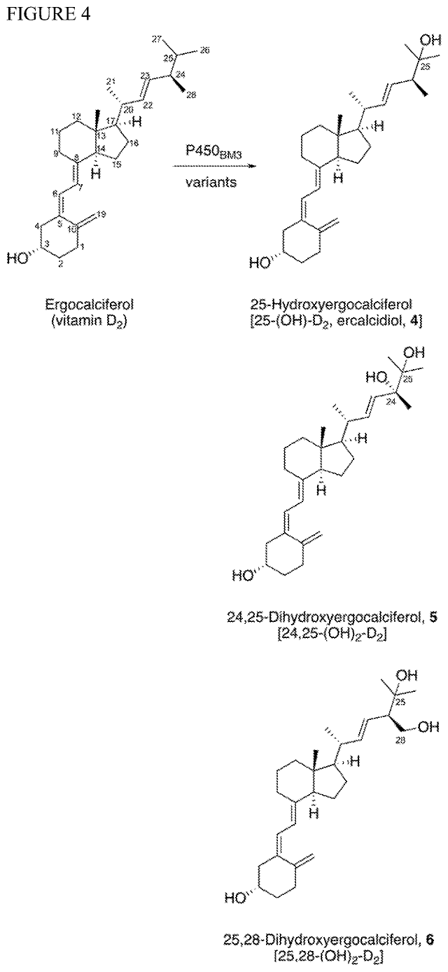

A secosteroid is a steroid with a broken ring, e.g. wherein a cycloalkyl ring in a traditional steroid has been broken. The secosteroid may be a 9,10-secosteroid, i.e. a secosteroid that may be obtainable by the cleavage of the bond between atoms C9 and C10 of the steroid B-ring. The secosteroid preferably comprises a carbon chain of at least 5 carbons in length at C17, more preferably a carbon chain of at least 6 carbons in length at C17, most preferably a carbon chain of 6 carbons in length at C17 and substituted at the fifth carbon of the chain with a methyl group. The secosteroid comprising a carbon chain of at least 5 carbons in length at C17 is preferably a 9,10-secosteroid. The fifth carbon of the carbon chain at C17 is referred to herein as “C25” (see e.g. FIGS. 1-4).

The secosteroid may be a vitamin D. The vitamin D may be selected from vitamin D₂ (ergocalciferol), vitamin D₃ (cholecalciferol), vitamin D₄ (22,23-dihydroergocalciferol), vitamin D₅ (sitocalciferol). Vitamin D₁ is a mixture of vitamin D₂ and lumisterol rather than a pure compound, and the term vitamin D₁ is no longer used. The vitamin D₂ may be present in a mixture of vitamin D₂ and lumisterol. The method of the invention may oxidise a secosteroid, such as a vitamin D, at C25. Preferably, the vitamin D is Vitamin D₂ or Vitamin D₃.

The process is typically for oxidising a vitamin D at C25. The process is typically selective for oxidising the vitamin D at C25 over oxidation at another position of the vitamin D, such as over oxidation at any other position of the vitamin D. The process may be selective for oxidising the vitamin D at C25 over oxidation at C20, C21, C22, C23, C24, C26 and/or C27 of the vitamin D. The process may produce at least 5-fold more product oxidised at C25 than a product oxidised at another position of the vitamin D, such as than oxidised at any other position of the vitamin D. The process may produce at least 10-fold more or at least 100-fold more product oxidised at C25 than a product oxidised at another position of the vitamin D, such as than oxidised at any other position of the vitamin D.

The process is typically carried out in the presence of the mutant CYP102A enzyme, the substrate and the natural co-factors of the wild-type enzyme, typically dioxygen and a reducing agent such as NADPH. The process may be carried out with an enzyme such as a dehydrogenase and its co-substrate to regenerate the NADPH from NADP⁺ with concomitant oxidation of the co-substrate of the dehydrogenase enzyme. The process may be carried out by regenerating the NADPH co-factor by electrochemical methods known to those in the art. Alternatively, the process may be carried out in the presence of hydrogen peroxide, e.g. wherein the heme monooxygenase domain comprises a mutation that allows the activation of the heme in the absence of a reductase domain, e.g. T268E of SEQ ID NO: 2.

The process may be carried out in the presence of an additional monooxygenase enzyme to oxidise the product produced by the mutant CYP102A enzyme at an additional position. The additional position may be at carbon 1 of the oxidised secosteroid produced by the mutant CYP102A enzyme. The additional monooxygenase enzyme may oxidise 25-hydroxy-vitamin D₃ to 1,25-dihydroxy vitamin D₃. The additional monooxygenase enzyme may oxidise 25-hydroxy-vitamin D₂ to 1,25-dihydroxy vitamin D₂. The additional monooxygenase enzyme may be a CYP27A enzyme, such as a CYP27A1, and/or a CYP27R enzyme, such as a CYP27R1.

In the process, the concentration of the mutant CYP102A enzyme is typically from 10⁻⁸ to 10⁻² M, preferably from 10⁻⁷ to 10⁻⁴ M. Generally the process is carried out at a temperature and/or pH at which the enzyme is functional, such as when the enzyme has at least 20%, 50%, 80% or more of peak activity. Typically the pH is from 3 to 11, such as 5 to 9 or 6 to 8, preferably 7 to 7.8 or 7.4. Typically the temperature is 10° C. to 90° C., such as 25° C. to 75° C. or 30° C. to 60° C.

The process may be carried out at an initial substrate:enzyme ratio of at least 100:1, such as at least 500:1, at least 1000:1, at least 5000:1 or at least 10000:1. The process may be carried out an initial substrate:enzyme ratio of between 100:1 and 100000:1, such as between 500:1 and 20000:1.

### Mutant CYP102A Enzymes

The mutant CYP102A enzyme comprises a heme monooxygenase domain comprising a P450 fold.

The enhanced monooxygenase activity and/or altered product selectivity may be assessed with respect to the corresponding wild-type CYP102A enzyme. The enhanced monooxygenase activity and/or altered product selectivity may be assessed with respect to SEQ ID NO: 2. The enhanced monooxygenase activity and/or altered product selectivity may be assessed with respect to the starting CYP102A enzyme prior to mutation.

Monooxygenases are enzymes that incorporate a single oxygen atom into substrates. Enhancing monooxygenase activity means to increase the rate at which a monooxygenase catalyses the incorporation of a single oxygen atom into substrates. The enhanced monooxygenase activity may be enhanced incorporation of a single oxygen atom into a secosteroid, more preferably a vitamin D, more preferably at the C25 of a vitamin D, and most preferably at the C25 of vitamin D₃ or vitamin D₂. The enhanced monooxygenase activity may be enhanced oxidation of vitamin D₃ to 25-hydroxy-vitamin D₃. The enhanced monooxygenase activity may be enhanced oxidation of vitamin D₂ to 25-hydroxy-vitamin D₃.

Enhanced monooxygenase activity may be characterised in terms of an increased conversion of substrate or an increased turnover number with one or more secosteroids for oxidation. The increased substrate conversion or increased turnover number may or may not be shared across all secosteroids utilised by the mutant CYP102A enzyme. The mutant CYP102A enzyme typically displays a substrate conversion which is at least 10%, 20%, 50%, 100%, 500%, 1000%, 2000%, 5000%, 10000% greater than that of the wild type enzyme. The mutant CYP102A enzyme may also have a turnover number which is at least 50%, 100%, 150%, 500%, 1000%, 2000%, 5000%, 10000% greater than that of the wild type enzyme.

The mutant CYP102A enzyme displays altered substrate specificity, allowing preferential utilization of secosteroids, whereas the wild type enzyme and known mutants are not able to oxidize secosteroids.

The mutant CYP102A enzyme display altered product selectivity, i.e. new products not formed at all by the wild type enzyme become the majority or dominant product. Further altered characteristics of the mutant enzymes and of the oxidation processes carried out by the mutant enzymes are described below. Typically, the new product features a single hydroxyl at C21 to C27 of a secosteroid, such as the C25 of a steroid, preferably at the C25 of a secosteroid, more preferably at the C25 of a vitamin D, and most preferably and the C25 of vitamin D₃ or vitamin D₂.

The mutant enzyme has altered substrate selectivity. Altered substrate selectivity is where a wild-type CYP102A enzyme has been modified to increase or decrease binding affinity for compounds at the active site of the enzyme. For example, the CYP102A enzyme may be modified to increase binding affinity for steroids, preferably secosteroids, more preferably vitamin Ds, and most preferably vitamin D₃ or vitamin D₂, at the active site of the enzyme. The CYP102A enzyme may be modified to decrease binding affinity for non-desired substrates, such as the natural substrates for the enzyme, fatty acids or steroids that are not 9,10-secosteroids, at the active site of the enzyme.

The heme monooxygenase domain comprising a P450 fold may comprise a sequence having at least 39.8% identity to amino acid residues 1-456 of SEQ ID NO: 2. Preferably, the heme monooxygenase domain comprising a P450 fold may comprise a sequence having at least 40%, 41.6%, 45%, 50%, 55%, 60%, 65%, 70%, 75%, 80%, 85%, 90%, 95%, 95%, 97% or 99% identity to amino acid residues 1-456 of SEQ ID NO: 2. More preferably, the heme monooxygenase domain comprising a P450 fold may comprise a sequence having at least 80%, 85%, 90%, 95%, 95%, 97% or 99% identity to amino acid residues 1-456 of SEQ ID NO: 2. The heme monooxygenase domain comprising a P450 fold may comprise amino acid residues 1-456 of SEQ ID NO: 2.

Amino acid residues 1-456 of SEQ ID NO:2 include the complete P450 fold of the monooxygenase domain. It has been suggested that the monooxygenase domain may be considered to be amino acid residues 1-470 of SEQ ID NO: 2 (see Peterson et al. Steroids, 1992, 62, 117-123). Accordingly, in some embodiments, the heme monooxygenase domain comprising a P450 fold may comprise a sequence having at least 39.8% identity to amino acid residues 1-470 of SEQ ID NO: 2. Preferably, the heme monooxygenase domain comprising a P450 fold may comprise a sequence having at least 40%, 41.6%, 45%, 50%, 55%, 60%, 65%, 70%, 75%, 80%, 85%, 90%, 95%, 95%, 97% or 99% identity to amino acid residues 1-470 of SEQ ID NO: 2. More preferably, the heme monooxygenase domain comprising a P450 fold may comprise a sequence having at least 80%, 85%, 90%, 95%, 95%, 97% or 99% identity to amino acid residues 1-470 of SEQ ID NO: 2. The heme monooxygenase domain comprising a P450 fold may comprise amino acid residues 1-470 of SEQ ID NO: 2.

The heme monooxygenase domain comprising a P450 fold may comprise a sequence having at least 39.8% identity to SEQ ID NO: 3, for example, the sequences as set out in SEQ ID NOs: 3-22. Preferably, the heme monooxygenase domain comprising a P450 fold may comprise a sequence having at least 40%, 41.6%, 45%, 50%, 55%, 60%, 65%, 70%, 75%, 80%, 85%, 90%, 95%, 95%, 97% or 99% identity to SEQ ID NO: 3. More preferably, the heme monooxygenase domain comprising a P450 fold may comprise a sequence having at least 80%, 85%, 90%, 95%, 95%, 97% or 99% identity to SEQ ID NO: 3. The heme monooxygenase domain comprising a P450 fold may comprise SEQ ID NO: 3.

Preferably, the mutant CYP102A enzyme is a fusion of the heme monooxygenase domain to a reductase domain.

The reductase domain typically transfers one or more electrons from a reducing agent, such as NADH or NADPH, to the heme of the monooxygenase domain comprising the P450 fold. The reductase domain may comprise one or more cofactors to shuttle one or more electrons from the reducing agent to the heme, such as FAD and FMN or a ferredoxin or a flavodoxin.

The reductase domain may comprise or consist of a naturally occurring reductase or a domain that has at least 40% identity with a naturally occurring reductase, such as at least 80%, 85%, 90%, 95%, 95%, 97% or 99% identity. The reductase domain may comprise or consist of a reductase domain of any electron transfer chain found in naturally occurring P450 systems.

The reductase domain may be a diflavin electron transfer domain, which contains both FAD and FMN prosthetic groups in a single polypeptide. The reductase domain may comprise or consist of a cytochrome P450 reductase (CPR) domain, such as a prokaryotic CPR domain or a eukaryotic CPR domain.

The reductase domain may comprise a flavin-dependent reductase domain, such as a putidaredoxin reductase. The reductase domain may comprise an FAD-containing reductase domain, such as a prokaryotic FAD-containing reductase domain, and a ferredoxin or flavodoxin. The reductase domain may comprise or consist of an electron transfer redoxin that is able to mediate the transfer of electrons from a reducing agent, such as NADPH, NADH or FADH, to the heme of the monooxygenase domain comprising a P450 fold. The electron transfer redoxin may be a naturally occurring electron transfer redoxin or a protein that has at least 40% identity with a naturally occurring electron transfer redoxin, such as at least 80%, 85%, 90%, 95%, 95%, 97% or 99% identity. The electron transfer redoxin is typically a redoxin of any electron transfer chain found in naturally occurring P450 enzyme systems. The electron transfer redoxin is typically a 2Fe-2S redoxin, such as putidaredoxin, or a flavodoxin.

The reductase domain may comprise a sequence having at least 40% identity to amino acid residues 471-1048 of SEQ ID NO: 2. Preferably, the reductase domain may comprise a sequence having at least 45%, 50%, 55%, 60%, 65%, 70%, 75%, 80%, 85%, 90%, 95%, 95%, 97% or 99% identity to amino acid residues 471-1048 of SEQ ID NO: 2. More preferably, the reductase domain may comprise a sequence having at least 80%, 85%, 90%, 95%, 95%, 97% or 99% identity to amino acid residues 471-1048 of SEQ ID NO: 2. The reductase domain may comprise amino acid residues 471-1048 of SEQ ID NO: 2.

It is understood that the reductase domain of CYP102A1 mediates the transfer of two electrons from NADPH to the heme in the P450 monooxygenase domain for the activation of dioxygen and formation of the ferryl, Compound I species that oxidises the substrate's C—H bond. The amino acid substitutions discussed herein typically alter the binding orientation of the substrate to present a specific target C—H bond to the ferryl species, leading to higher activity and selectivity. These substitutions do not affect the mechanism of the formation of the ferryl species. Accordingly, pathways could be utilised to generate the ferryl species of a mutant CYP102A enzyme variant with the required product selectivity and provide substrate oxidation without the reductase domain, or NADPH or oxygen being present. One example is the conversion of CYP102A1 to a peroxygenase by the mutation T268E, such that the heme with the iron centre in the Fe(III) state can be converted to the ferryl species with hydrogen peroxide to effect substrate oxidation—the reductase domain, NADPH and dioxygen are not required (Shoji et al., Catal. Sci. Technol. 2016, 6, 5806-5811). Other substitutions such as T268D and T268H also allow the CYP102A1 enzyme to function as a peroxygenase. Addition of the T268E mutation to the heme domain of the mutant CYP102A enzymes disclosed herein having secosteroid oxidation activity would provide an alternative system for oxidising vitamin D₂ to 25-hydroxy-vitamin D₂ and oxidising vitamin D₃ to 25-hydroxy-vitamin D₃ without a reductase domain.

Accordingly, in some aspects the mutant CYP102A enzyme does not comprise a reductase domain. In such aspects, the process is carried out in the presence of a mechanism to form the ferryl species in the heme monooxygenase domain, for example, in the presence of hydrogen peroxide or in the presence of a separate polypeptide encoding a reductase domain as described herein. The mutant CYP102A enzyme may additionally comprise the substitution T268E/T268D/T268H in SEQ ID NO:2 or a corresponding substitution in the polypeptide chain of a wild-type CYP102A enzyme at one or more positions corresponding to amino acid residue 268 of SEQ ID NO: 2. Said substitution may be in addition to any of the other substitutions discussed herein.

The mutant CYP102A enzyme may be a mutant enzyme selected from CYP102A1, CYP102A2, CYP102A3, CYP102A4, CYP102A5, CYP102A6, CYP102A7, CYP102A8, CYP102A9, CYP102A10, CYP102A11, CYP102A12, CYP102A13, CYP102A14, CYP102A15, CYP102A16, CYP102A25, CYP102A26, Krac9955 and Krac0936, as set out in SEQ ID NOs: 3-22, respectively. Preferably, the mutant CYP102A enzyme is a mutant CYP102A1 enzyme. The CYP102A1 enzyme may be a natural or artificial homologue of CYP102A1, for example, having at least 40% amino acid identity to SEQ ID NO: 2. More preferably, the mutant CYP102A1 enzyme have comprise a sequence having at least 45%, 50%, 55%, 60%, 65%, 70%, 75%, 80%, 85%, 90%, 95%, 95%, 97% or 99% identity to SEQ ID NO: 2. Said homologues typically comprise amino acid sequences which correspond to (i.e. are homologous to or the same as) amino acid sequences in the heme monooxygenase domain of CYP102A1 (represented by amino acid positions 1-456 of SEQ ID NO: 2; or in some embodiments represented by amino acid positions 1-470 of SEQ ID NO:2).

The CYP102A1 enzyme may comprise (or consist of) a sequence which has at least 40% identity to SEQ ID NO: 2 (the sequence of CYP102A1). The CYP102A1 enzyme may have any of the specified percentage homologies when compared to amino acid residues 1 to 456 of SEQ ID NO: 2. The CYP102A1 enzyme may have any of the specified percentage homologies when compared to amino acid residues 1 to 470 of SEQ ID NO: 2. The homologous sequence may represent a mutated portion of the CYP102A1 sequence and/or may be present in the form of the full-length fusion disclosed herein.

Homology can be measured using known methods. For example, the UWGCG Package provides the BESTFIT program which can be used to calculate homology (for example used on its default settings) (Devereux et al. Nucleic Acids Res. 1984, 12, 387-395). The PILEUP and BLAST algorithms can be used to calculate homology or line up sequences (typically on their default settings), for example, as described in Altschul S. F. (J. Mol. Evol. 1993, 36, 290-300) and in Altschul, S. F. et al. (J. Mol. Biol. 1990, 215, 403-410).

Software for performing BLAST analyses is publicly available through the National Center for Biotechnology Information (http://www.ncbi.nlm.nih.gov/).

Typically, the mutant CYP102A enzyme may comprise at least 2, 5, 10, 20, 40, 60, 80, 100, 120, 140, 160, 180, 200 or 220 mutations (each of which can be substitutions, insertions or deletions) when compared to amino acid residues 1-456 of SEQ ID NO:2. The mutant CYP102A enzyme may comprise no more than 2, 5, 10, 20, 40, 60, 80, 100, 120, 140, 160, 180, 200 or 220 mutations (each of which can be substitutions, insertions or deletions) when compared to amino acid residues 1-456 of SEQ ID NO:2. The mutant CYP102A enzyme may comprise at least 2, 5, 10, 20, 40, 60, 80, 100, 120, 140, 160, 180, 200 or 220 mutations (each of which can be substitutions, insertions or deletions) when compared to SEQ ID NO:2. The mutant CYP102A enzyme may comprise no more than 2, 5, 10, 20, 40, 60, 80, 100, 120, 140, 160, 180, 200 or 220 mutations (each of which can be substitutions, insertions or deletions) when compared to SEQ ID NO:2.

The mutant CYP102A enzyme may comprise at least 2, 5, 10, 20, 40, 60, 80, 100, 120, 140, 160, 180, 200 or 220 mutations (each of which can be substitutions, insertions or deletions) when compared to amino acid residues 1-470 of SEQ ID NO:2. The mutant CYP102A enzyme may comprise no more than 2, 5, 10, 20, 40, 60, 80, 100, 120, 140, 160, 180, 200 or 220 mutations (each of which can be substitutions, insertions or deletions) when compared to amino acid residues 1-470 of SEQ ID NO:2.

The enzymatic activity of the CYP102A enzyme of the invention is typically measured in vitro using any of the substrates or conditions mentioned herein and is typically given as the conversion rate (Conv; the percentage of substrate converted to products) and/or preferably, the turnover number (TON; the turnover number of the enzyme for producing the oxidised secosteroid). The conversion rate and/or turnover number is preferably measured when the secosteroid is vitamin D₂ or vitamin D₃ and/or wherein the product is 25-hydroxy-vitamin D₂ or 25-hydroxy-vitamin D₃, respectively. The conversion rate is typically measured at a substrate:enzyme ratio of e.g. 500:1 or 1000:1. The mutant CYP102A enzyme (for example when used in the process of the invention) may have a conversion rate of at least 10%, such as at least 15%, at least 20%, at least 25%, at least 30%, at least 35%, at least 40%, at least 45%, at least 50%, at least 55%, at least 60%, at least 65%, at least 70%, at least 75%, or at least 80% or more. The mutant CYP102A enzyme may have a turnover number of at least 50, such as at least 100, at least 200, at least 300, at least 400, at least 500, at least 600, at least 700 or at least 800 or more. The mutant CYP102A enzyme may have a conversion rate of at least 10% and a TON of at least 50.

The mutant CYP102A enzyme may comprise a sequence having at least 40% identity to SEQ ID NO: 2. Preferably, the mutant CYP102A enzyme may comprise a sequence having at least 45%, 50%, 55%, 60%, 65%, 70%, 75%, 80%, 85%, 90%, 95%, 95%, 97% or 99% identity to SEQ ID NO: 2. More preferably, the mutant CYP102A enzyme may comprise a sequence having at least 80%, 85%, 90%, 95%, 95%, 97% or 99% identity to SEQ ID NO: 2. The mutant CYP102A enzyme may comprise SEQ ID NO: 2.

The mutant CYP102A enzyme comprises substitution(s) in the polypeptide chain of a wild-type CYP102A enzyme at one or more positions, thereby enhancing monooxygenase activity and/or altering product selectivity of the mutant enzyme.

The mutant CYP102A enzyme preferably comprises a substitution at one or more positions corresponding to amino acid positions 435, 82, 184, 260 and/or 72 of SEQ ID NO: 2. Typically, the mutant CYP102A enzyme may comprise 2 or more, 3 or more, 4 or more or all 5 of the substitutions at positions corresponding to amino acid positions 435, 82, 184, 260 and/or 72 of SEQ ID NO: 2.

The one or more substitutions may be selected from E435I, E435M, E435T, A82M, A82L, A82I, A82F, A82W, A184I, T260G, T260A, S72A and/or S72G in SEQ ID NO:2, or corresponding substitutions in the polypeptide chain of a wild-type CYP102A enzyme at one or more positions corresponding to amino acid residues 435, 82, 184, 260 and/or 72 of SEQ ID NO: 2. Preferably, the one or more substitutions comprises E435I, E435M, E435T, S72A and/or S72G in SEQ ID NO:2. More preferably, the one or more substitutions comprise E435I or E435M in SEQ ID NO: 2.

The CYP102A enzyme may have 1, 2, 3, 4, 5 to 10, 10 to 20, 20 to 40 or more additional mutations, such as substitutions, insertions or deletions. These additional mutations may or may not enhance monooxygenase activity and/or alter product selectivity of the mutant CYP102A enzyme. The other mutations may be in the active site or outside the active site. For example, the mutations may be in the second sphere, i.e. residues which affect or contact the position or orientation of one or more of the amino acids in the active site. An insertion will typically be N and/or C terminal. Thus the enzyme may contain a short peptide of up to 20 amino acids or a full-length protein fused to either or both of the termini, e.g. to aid protein purification by affinity chromatography or immobilisation on a solid matrix, such as via a histidine tag. A deletion typically comprises the deletion of amino acids which are not involved in catalysis, such as those outside the active site (thus the enzyme is a mutated fragment of a naturally occurring enzyme).

Other mutations in the active site may alter the position and/or conformation of the substrate when it is bound in the active site. The mutations may make the site on the substrate which is to be oxidized more accessible to the heme group. Thus the mutations may be substitutions to an amino acid which has a smaller or larger, or more or less polar, side chain.

Preferably, the CYP102A enzyme additionally comprises one or more substitutions at one or more positions corresponding to amino acid residue positions 29, 78, 87, 178, 263, 264, 268, 328 and/or 354 of SEQ ID NO:2. The CYP102A enzyme may comprise 2 or more, 3 or more, 4 or more or 5 or more of the substitutions at positions corresponding to amino acid positions 78, 87, 178, 263, 264, 268, 328 and/or 354 of SEQ ID NO: 2. The one or more additional substitutions may be selected from V78F, F87A, F87V, F87I, V178F, V178W, V178L, V178I, I263G, A264G, T268S, A328G, and/or M354F or corresponding substitutions at one or more positions corresponding to amino acid residue positions 78, 87, 178, 263, 264, 268, 328 and/or 354 of SEQ ID NO: 2.

The CYP102A enzyme may comprise a said substitution at position 82 of SEQ ID NO: 2, or a position corresponding thereto, and a said substitution at position 87 of SEQ ID NO: 2 or a position corresponding thereto. The CYP102A enzyme may comprise one or more further mutations at position 184 of SEQ ID NO: 2, or a position corresponding thereto, a said substitution at position 260 of SEQ ID NO:2 or a position corresponding thereto, a said substitution at position 72 of SEQ ID NO:2 or a position corresponding thereto and/or a said substitution at position 435 of SEQ ID NO:2 or a position corresponding thereto. Preferably, the mutant CYP102A enzyme comprises substitutions at position 82 of SEQ ID NO:2, or a position corresponding thereto, at position 87 of SEQ ID NO:2, or a position corresponding thereto, and, at position 435 of SEQ ID NO:2 or a position corresponding thereto.

The CYP102A enzyme may comprise one or the following groups of substitutions in SEQ ID NO:2:
A82M/F87A;
A82M/F87I;
A82M/F87V;
A82M/F87S;
A82M/F87T;
A82M/T260G;
A82M/A184I;
A82M/S72A;
A82M/E435I, E435M or E435T;
A82M/E435I;
A82M/E435M;
F87A/T260G;
F87I/T260G;
F87V/T260G;
F87S/T260G;
F87T/T260G;
F87A/A184I;
F87I/A184I;
F87V/A184I;
F87S/A184I;
F87T/A184I;
F87A/S72A;
F87I/S72A;
F87V/S72A;
F87S/S72A;
F87T/S72A;
A82M/F87A/T260G;
A82M/F87I/T260G;
A82M/F87V/T260G;
A82M/F87S/T260G;
A82M/F87T/T260G;
A82M/F87A/A184I;
A82M/F87I/A184I;
A82M/F87V/A184I;
A82M/F87S/A184I;
A82M/F87T/A184I;
A82M/F87A/E435I, E435M or E435T;
A82M/F87I/E435I, E435M or E435T;
A82M/F87V/E435I, E435M or E435T;
A82M/F87S/E435I, E435M or E435T;
A82M/F87T/E435I, E435M or E435T;
A82M/F87A/E435I;
A82M/F87A/E435M;
A82M/F87I/E435I;
A82M/F87I/E435M;
A82M/F87V/E435I;
A82M/F87V/E435M;
A82M/F87S/E435I;
A82M/F87S/E435M;
A82M/F87T/E435I;
A82M/F87T/E435M;
F87A/A184I/T260G;
F87I/A184I/T260G;
F87V/A184I/T260G;
F87S/A184I/T260G;
F87T/A184I/T260G;
F87A/A184I/S72A;
F87I/A184I/S72A;
F87V/A184I/S72A;
F87S/A184I/S72A;
F87T/A184I/S72A;
F87A/A184I/E435I, E435M or E435T;
F87I/A184I/E435I, E435M or E435T;
F87V/A184I/E435I, E435M or E435T;
F87S/A184I/E435I, E435M or E435T;
F87T/A184I/E435I, E435M or E435T;
F87A/A184I/E435I;
F87A/A184I/E435M;
F87I/A184I/E435I;
F87I/A184I/E435M;
F87V/A184I/E435I;
F87V/A184I/E435M;
F87S/A184I/E435I;
F87S/A184I/E435M;
F87T/A184I/E435I;
F87T/A184I/E435M;
F87A/A82M/A184I/T260G;
F87I/A82M/A184I/T260G;
F87V/A82M/A184I/T260G;
F87S/A82M/A184I/T260G;
F87T/A82M/A184I/T260G;
F87A/A82M/T260G/S72A;
F87I/A82M/T260G/S72A;
F87V/A82M/T260G/S72A;
F87S/A82M/T260G/S72A;
F87T/A82M/T260G/S72A;
F87A/A82M/A184I/T260G/S72A;
F87I/A82M/A184I/T260G/S72A;
F87V/A82M/A184I/T260G/S72A;
F87S/A82M/A184I/T260G/S72A;
F87T/A82M/A184I/T260G/S72A;
1 F87A, F87I, F87V, F87S or F87T/A82M/A184I/T260G/E435I, E435M or E435T;
F87A/A82M/A184I/T260G/E435I;
F87A/A82M/A184I/T260G/E435M;
F87I/A82M/A184I/T260G/E435I;
F87I/A82M/A184I/T260G/E435M;
F87V/A82M/A184I/T260G/E435I;
F87V/A82M/A184I/T260G/E435M;
F87S/A82M/A184I/T260G/E435I;
F87S/A82M/A184I/T260G/E435M;
F87T/A82M/A184I/T260G/E435I;
F87T/A82M/A184I/T260G/E435M;
F87A, F87I, F87V, F87S or F87T/A82M/A184I/T260G/S72A/E435I, E435M or E435T;
F87A/A82M/A184I/T260G/S72A/E435I;
F87A/A82M/A184I/T260G/S72A/E435M;
F87I/A82M/A184I/T260G/S72A/E435I;
F87I/A82M/A184I/T260G/S72A/E435M;
F87V/A82M/A184I/T260G/S72A/E435I;
F87V/A82M/A184I/T260G/S72A/E435M;
F87S/A82M/A184I/T260G/S72A/E435I;
F87S/A82M/A184I/T260G/S72A/E435M;
F87T/A82M/A184I/T260G/S72A/E435I;
F87T/A82M/A184I/T260G/S72A/E435M;
F87A, F87I, F87V, F87S or F87T/A82M/A184I/T260G/S72A/E435I, E435M or E435T/L29M;
F87A/A82M/A184I/T260G/S72A/E435I/L29M;
F87I/A82M/A184I/T260G/S72A/E435I/L29M;
F87V/A82M/A184I/T260G/S72A/E435I/L29M;
F87S/A82M/A184I/T260G/S72A/E435I/L29M;
F87T/A82M/A184I/T260G/S72A/E435I/L29M;
F87A/A82M/A184I/T260G/S72A/E435M/L29M;
F87I/A82M/A184I/T260G/S72A/E435M/L29M;
F87V/A82M/A184I/T260G/S72A/E435M/L29M;
F87S/A82M/A184I/T260G/S72A/E435M/L29M;
F87T/A82M/A184I/T260G/S72A/E435M/L29M;
F87A/A82M/A184I/T260G/S72A/E435T/L29M;
F87I/A82M/A184I/T260G/S72A/E435T/L29M;
F87V/A82M/A184I/T260G/S72A/E435T/L29M;
F87S/A82M/A184I/T260G/S72A/E435T/L29M;
1 F87T/A82M/A184I/T260G/S72A/E435T/L29M;
or corresponding substitutions in the polypeptide chain of a wild-type CYP102A enzyme at one or more positions corresponding to the amino acid residue positions of SEQ ID NO: 2 as listed above.

The CYP102A enzyme may, in addition to any of the combinations of mutations described above, additionally comprise one or more substitutions at one or more positions corresponding to amino acid residue positions 47, 51, 74, 171, 188, 239, 259, 307, 319 and/or 353 of SEQ ID NO:2. The one or more substitutions may be selected from R47L, Y51F, A74G, H171L, N239H, I259V, L188Q, Q307H, N319Y and/or L353I in SEQ ID NO:2, or corresponding substitutions in the polypeptide chain of a wild-type CYP102A enzyme at one or more positions corresponding to amino acid residue positions 47, 51, 74, 171, 188, 239, 259, 307, 319 and/or 353 of SEQ ID NO: 2.

Where specific mutants of CYP102A are described, the letter of the amino acid residue present in the wild-type form of CYP102A is followed by the position, followed by the amino acid in the mutant. These positions can be correlated to the numbering shown in SEQ ID NO:2. To denote multiple mutations in the same protein each mutation is listed separated by slashes. Particularly preferred mutants may be described using the entry numbers provided in tables 1 to 5 of the examples.

While mutations are defined by reference to a position in CYP102A1, the invention also encompasses equivalent substitution mutations at a homologous or corresponding position in the polypeptide chain of a homologue of CYP102A which shares at least 39.8% amino acid identity to SEQ ID NO:2. An equivalent position is determined by reference to amino acids 1-456 of SEQ ID NO:2. The homologous or corresponding position can be readily deduced by lining up the sequence of the homologue and the sequence amino acids 1-456 of CYP102A1 (SEQ ID NO:2) based on the homology between the sequences. The PILEUP and BLAST algorithms can be used to line up the sequences. Where the homologous or corresponding amino acid referred to is an active site residue, it will generally be in a similar place in the active site of the homologue as any of the specific amino acids discussed herein.

Despite having a highly conserved tertiary structure, the P450 superfamily of enzymes is well known to those skilled in the art to be unusual among proteins and enzymes in having primary structures with low homology. P450 enzymes in different families have amino acid identities as low as 20%. A sample of alignment between CYP102A1 and structurally characterized P450 enzymes is shown in Table A. In the systematic classification of the P450 superfamily, enzymes with just 40% amino acid identity are placed within the same family, and closely related members of a family (>55% identity) are grouped into sub-families (see, for example, Table B). It is increasingly recognized that the 40% cut-off for assigning enzymes to the same family could be too high in some instances, and that enzymatic activity and the higher homology often observed for active site residues may need to be taken more into consideration in future.

Thus, enzymes that comprise a sequence having at least 40% amino acid identity to CYP102A1 (SEQ ID NO:2) are also be readily identifiable on the basis of the “P450 fold”, and alignment of sequences of homologues to introduce an equivalent mutation at a corresponding or homologous position may be assisted by knowledge of the conserved nature of the arrangement of α helices and β strands that comprises the P450 fold shared throughout the enzyme family.

TABLE A
Sequence similarities between CYP102A1 heme domain
(amino acid residues 1-470) and various structurally
characterized cytochrome P450 enzymes.

| Cytochrome | CYP102A1 | | |
|---|---|---|---|
| P450 | Identities | Positives | Gaps |
| CYP505 | 188/452 (41%) | 268/452 (59%) | 10/452 (2%) |
| (P450foxy) | | | |
| CYP3A4 | 114/395 (28%) | 184/395 (45%) | 37/395 (9%) |
| CYP51 | 100/410 (24%) | 180/410 (43%) | 27/410 (6%) |
| (M. tuberculosis) | | | |
| P4502R1 | 76/252 (30%) | 126/252 (50%) | 19/252 (7%) |
| CYP175A1 | 98/364 (26%) | 150/364 (41%) | 61/364 (16%) |
| CYP2D6 | 59/232 (25%) | 96/232 (41%) | 24/232 (10%) |
| CYP2A6 | 99/434 (22%) | 174/434 (40%) | 26/434 (5%) |
| CYP108A1 | 58/219 (26%) | 97/219 (44%) | 30/219 (13%) |
| (P450terp) | | | |
| CYP2A13 | 99/437 (22%) | 170/437 (38%) | 26/437 (5%) |
| CYP2C8 | 57/198 (28%) | 85/198 (42%) | 7/198 (3%) |
| CYP107L1 | 6/236 (27%) | 102/236 (43%) | 52/236 (22%) |
| (P450pikC) | | | |
| CYP2B4 | 55/229 (24%) | 102/229 (44%) | 13/229 (5%) |
| CYP2C9 | 51/177 (28%) | 80/177 (45%) | 6/177 (3%) |
| CYP2C5 | 49/165 (29%) | 76/165 (46%) | 6/165 (3%) |
| CYP165B3 | 75/324 (23%) | 127/324 (39%) | 63/324 (19%) |
| (P450oxyB) | | | |
| CYP154C1 | 59/216 (27%) | 84/216 (38%) | 38/216 (17%) |
| CYP154A1 | 70/343 (20%) | 138/343 (40%) | 53/343 (15%) |
| CYP245A1 | 47/179 (26%) | 74/179 (41%) | 34/179 (18%) |
| CYP119A1 | 51/180 (28%) | 80/180 (44%) | 45/180 (25%) |
| CYP8A1 | 46/157 (29%) | 72/157 (45%) | 24/157 (15%) |
| CYP167A1 | 51/219 (23%) | 94/219 (42%) | 38/219 (17%) |
| (P450epoK) | | | |
| CYP107A1 | 65/283 (22%) | 114/283 (40%) | 36/283 (12%) |
| (P450eryF) | | | |
| CYP199A2 | 43/176 (24%) | 73/176 (41%) | 27/176 (15%) |
| CYP101A1 | 56/223 (25%) | 90/223 (40%) | 57/223 (25%) |
| (P450cam) | | | |
| CYP165C1 | 48/204 (23%) | 90/204 (44%) | 41/204 (20%) |
| (P450oxyC) | | | |
| CYP119A2 | 44/166 (26%) | 70/166 (42%) | 35/166 (21%) |
| CYP152A1 | 41/148 (27%) | 62/148 (41%) | 20/148 (13%) |
| (P450BSβ) | | | |
| CYP121 | 44/184 (23%) | 72/184 (39%) | 35/184 (19%) |

TABLE B
Sequence similarities between the CYP102A1 heme domain (amino
acid residues 1-470 of SEQ ID NO:2) and those of other CYP102A
subfamily members. Note that, despite being in the same
subfamily, CYP102A25 is only 39.8% homologous to CYP102A1,
this homology being almost the same as between CYP102Al
and CYP102B1 from another CYP102 sub-family.

| Cytochrome | CYP102A1 | | |
|---|---|---|---|
| P450 | Identities | Positives | Gaps |
| CYP102A2 | 63.5% | 75.5% | 5.2% |
| CYP102A3 | 64.3% | 77.7% | 2.3% |
| CYP102A4 | 62.6% | 76.6% | 6.1% |
| CYP102A5 | 65.2% | 78.8% | 2.8% |
| CYP102A6 | 50.3% | 66.0% | 3.6% |
| CYP102A7 | 64.5% | 78.7% | 2.3% |
| CYP102A8 | 63.0% | 76.8% | 6.1% |
| CYP102A9 | 63.9% | 77.2% | 6.1% |
| CYP102A10 | 51.9% | 67.6% | 3.2% |
| CYP102A11 | 52.9% | 68.2% | 2.8% |
| CYP102A12 | 50.0% | 64.9% | 5.6% |
| CYP102A13 | 48.9% | 64.9% | 4.8% |
| CYP102A14 | 48.9% | 64.9% | 4.8% |
| CYP102A15 | 58.5% | 74.3% | 2.3% |
| CYP102A16 | 63.0% | 76.8% | 6.1% |
| CYP102A18 | 44.9% | 61.1% | 6.9% |
| (Krac9955) | | | |
| CYP102A18 | 54.1% | 71.1% | 2.8% |
| (Krac0936) | | | |
| CYP102A25 | 39.8% | 57.7% | 6.5% |
| CYP102A26 | 64.1% | 77.5% | 4.8% |
| CYP102B1 | 39.7% | 53.6% | 17.5% |

It is to be understood that members of the CYP102A family are fusions of an electron transfer reductase domain and a heme monooxygenase domain. These domains may be cleaved proteolytically or by truncation of the full-length gene. The active site (substrate binding pocket) is located in the heme domain. Some members of the CYP102 family are not fusion proteins but the sequence homology with the CYP102A heme domain is 40%. Thus, sequence homology may be measured solely over the heme domain in these circumstances. Equivalent residues in these enzymes to those in CYP102A disclosed herein can be identified by sequence homology and structural analysis known to those skilled in the art.

An amino acid in the active site is one which lines or defines the site in which the substrate is bound during catalysis or one which lines or defines a site through which the substrate must pass before reaching the catalytic site. Therefore such an amino acid typically interacts with the substrate during entry to the catalytic site or during catalysis. Such an interaction typically occurs through an electrostatic interaction (between charged or polar groups), hydrophobic interaction, hydrogen bonding or van der Waals forces. Active site amino acids can be identified by sequence alignment and reference to the known crystal structure of the heme domain of wild type CYP102A, or the crystal structure of the homologues.

Where the mutated residue is not an active site residue, computerized or manual alignment of sequences of the homologue and of CYP102A1 may be carried out to deduce the homologous or corresponding position, which may be assisted by knowledge of the residues flanking the mutated position in CYP102A1 set out in SEQ ID NO:2. Thus, for example, the 10 N-terminally and C-terminally flanking residues to the following positions in CYP102A1 are:

CDESRFDKNL(S72)QALKFVRDFA
DKNLSQALKF(V78)RDFAGDGLFT
SQALKFVRDF(A82)GDGLFTSWTH
FVRDFAGDGL(F87)TSWTHEKNWK
DQPHPFITSM(V178)RALDEAMNKL
ITSMVRALDE(A184)MNKLQRANPD
DDENIRYQII(T260)FLIAGHETTS
NIRYQIITFL(I263)AGHETTSGLL
IRYQIITFLI(A264)GHETTSGLLS
IITFLIAGHE(T268)TSGLLSFALY
LNEALRLWPT(A328)PAFSLYAKED
EYPLEKGDEL(M354)VLIPQLHRDK
DHTNYELDIK(E435)TLTLKPEGFV

Conservation of 2, 3 or more of the N- and/or C-terminal flanking residues can allow for deduction of the homologous or corresponding position at which a mutation is to be introduced.

Similar analyses can be carried out for any other positions in CYP102A1 that are referred to in the description so as to identify the homologous or corresponding site in a naturally occurring homologue.

The nature of the amino acid to be substituted at the positions of CYP102A described herein (or equivalent positions as defined above) is primarily determined by the requirement for the mutant to display an enhanced monooxygenase activity and/or altered product specificity. Thus, an amino acid that is introduced will typically enhance monooxygenase activity and/or alter product specificity. Where any reference is made to specific substitutions in CYP102A, it is to be understood that any substitution of another amino acid residue at the same position which has effects which are redundant over, or similar to, the effect of the specific substitution on the oxidation activity and/or product specificity of the CYP102A enzyme, is encompassed according to the present invention. Similarly, where a specific substitution also has an effect on another parameter of the CYP102A enzyme, such as substrate specificity, it is to be understood that substitutions of other amino acid residues that also elicit a redundant or similar effect are also contemplated for use according to the invention.

In some embodiments, the substitution introduces a conservative change, which replaces the amino acid with another amino acid of similar chemical structure, similar chemical properties or similar side-chain volume. The amino acids introduced may have similar polarity, hydrophilicity or hydrophobicity to the amino acids they replace. Conservative amino acid changes are well known in the art and may be selected in accordance with the changes defined in table C. Where amino acids have similar polarity, this can also be determined by reference to the hydropathy scale for amino acid side chains (table D). The side chain volumes of the twenty naturally occurring amino acids may be grouped (from smallest to largest) as follows: Gly, Ala, Ser, Cys, Thr≈Asp≈Pro≈Asn, Val≈Glu≈Gln≈His, Ile≈Leu≈Met≈Lys, Phe≈Arg≈Tyr≈Trp.

TABLE C
Physical characteristics of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged (+) | Trp | aromatic, hydrophobic, neutral |
| Leu | aliphatic, hydrophobic, neutral | Tyr | aromatic, polar, hydrophobic |

TABLE D
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Conservative amino acid changes may also be determined by reference to the Point Accepted Mutation (PAM) or BLOcks Substitution Matrix (BLOSUM) family of scoring matrices for conservation of amino acid sequence. Thus, conservative amino acid changes may be members of an equivalence group, being a set of amino acids having mutually positive scores in the similarity representation of the scoring matrix selected for use in an alignment of the reference and mutant polypeptide chains.

It is to be understood that the definitions of physical characteristics provided in Table C are not considered to be limiting on the invention. For example, the amino acid proline is classified as non-polar but it also has the property of being rigid and can cause changes in secondary structure. For example prolines are often found at the end of helices. Also, depending on the specific context of the side chain of a given amino acid residue, for example the amino acid tyrosine, generally classed as hydrophobic due to its aromatic ring, may have analogous functional effects to a polar amino acid residue such as threonine via its hydroxyl group. Thus, tyrosine may be considered to be both a hydrophobic and a polar amino acid for the purposes of the invention. Furthermore, amino acids which are described as polar or hydrophilic may be uncharged or charged, and may also be basic or acidic. The amino acid histidine is well known to have a pKa value near 7, so that at neutral pH depending upon the protein environment, it may or not be protonated on its side chain, and thus may or not carry a charge. Thus, histidine may be considered to be both a polar charged or a polar uncharged amino acid residue for the purposes of the invention.

The substitution in the polypeptide chain at a position corresponding to amino acid residue 435 of SEQ ID NO: 2 may be a substitution with a neutral amino acid. Preferably, the substitution in the polypeptide chain at a position corresponding to amino acid residue 435 of SEQ ID NO: 2 may be a substitution with a hydrophobic, neutral amino acid. The substitution in the polypeptide chain at a position corresponding to amino acid residue 435 of SEQ ID NO: 2 may be a substitution selected from E435I, E435M and E435T, preferably E435I and E435M, more preferably E435I.

The substitution in the polypeptide chain at a position corresponding to amino acid residue 82 of SEQ ID NO: 2 may be a substitution with a neutral and/or hydrophobic amino acid. Residue 82 of SEQ ID NO:2 lies at the far end of the substrate binding pocket from the heme. Preferably, the substitution in the polypeptide chain at a position corresponding to amino acid residue 82 of SEQ ID NO: 2 may be a substitution with a residue that blocks off the far end of the substrate binding pocket from the heme, such as a substitution that increases the side chain volume of the residue at position 82. The substitution in the polypeptide chain at a position corresponding to amino acid residue 82 of SEQ ID NO: 2 may be a substitution selected from A82M, A82L, A82I, A82F and A82W.

The substitution in the polypeptide chain at a position corresponding to amino acid residue 184 of SEQ ID NO: 2 may be a substitution with an aliphatic, hydrophobic and/or neutral amino acid. The substitution in the polypeptide chain at a position corresponding to amino acid residue 184 of SEQ ID NO: 2 may be A184I.

The substitution in the polypeptide chain at a position corresponding to amino acid residue 260 of SEQ ID NO: 2 may be a substitution with an aliphatic, hydrophobic and/or neutral amino acid. Preferably, the substitution in the polypeptide chain at a position corresponding to amino acid residue 260 of SEQ ID NO: 2 may be a substitution that reduces the side chain volume of the residue at position 260, such as S, C, A or G. The substitution in the polypeptide chain at a position corresponding to amino acid residue 260 of SEQ ID NO: 2 may be T260A or T260G.

The substitution in the polypeptide chain at a position corresponding to amino acid residue 72 of SEQ ID NO: 2 may be a substitution with an aliphatic, hydrophobic and/or neutral amino acid. Preferably, the substitution in the polypeptide chain at a position corresponding to amino acid residue 72 of SEQ ID NO: 2 may be a substitution that reduces the side chain volume of the residue at position 72. The substitution in the polypeptide chain at a position corresponding to amino acid residue 72 of SEQ ID NO: 2 may be S72A or S72G.

The substitution in the polypeptide chain at a position corresponding to amino acid residue 78 of SEQ ID NO: 2 may be a substitution with an aromatic, hydrophobic and/or neutral amino acid. Preferably, the substitution in the polypeptide chain at a position corresponding to amino acid residue 78 of SEQ ID NO: 2 may be a substitution that increases the side chain volume of the residue at position 78. The substitution in the polypeptide chain at a position corresponding to amino acid residue 78 of SEQ ID NO: 2 may be V78F.

The substitution in the polypeptide chain at a position corresponding to amino acid residue 87 of SEQ ID NO: 2 may be a substitution with an aliphatic, hydrophobic and/or neutral amino acid. Preferably, the substitutions in the polypeptide chain at a position corresponding to amino acid residue 87 of SEQ ID NO: 2 is any substitution that enables oxidation of steroid substrates, e.g. A/V/I/S/T. The substitution in the polypeptide chain at a position corresponding to amino acid residue 87 of SEQ ID NO: 2 may be selected from F87A, F87V and F87I. Preferably, the mutant CYP102A enzyme comprises one or more of the substitutions disclosed herein in combination with a substitution in the polypeptide chain at a position corresponding to amino acid residue 87 of SEQ ID NO: 2 with an aliphatic and neutral amino acid, such as a substitution selected from F87A, F87V, F87I, F87S and F87T.

The substitution in the polypeptide chain at a position corresponding to amino acid residue 178 of SEQ ID NO: 2 may be a substitution with a hydrophobic and/or neutral amino acid. Preferably, the substitution in the polypeptide chain at a position corresponding to amino acid residue 178 of SEQ ID NO: 2 may be a substitution that increases the side chain volume of the residue at position 178. The substitution in the polypeptide chain at a position corresponding to amino acid residue 178 of SEQ ID NO: 2 may be selected from V178F, V178W, V178L and V178I.

The substitution in the polypeptide chain at a position corresponding to amino acid residue 263 of SEQ ID NO: 2 may be a substitution with a aliphatic and/or neutral amino acid. Preferably, the substitution in the polypeptide chain at a position corresponding to amino acid residue 263 of SEQ ID NO: 2 may be a substitution that decreases the side chain volume of the residue at position 263. The substitution in the polypeptide chain at a position corresponding to amino acid residue 263 of SEQ ID NO: 2 may be 1263G.

The substitution in the polypeptide chain at a position corresponding to amino acid residue 264 of SEQ ID NO: 2 may be a substitution with a aliphatic and/or neutral amino acid. Preferably, the substitution in the polypeptide chain at a position corresponding to amino acid residue 263 of SEQ ID NO: 2 may be a substitution that decreases the side chain volume of the residue at position 263. The substitution in the polypeptide chain at a position corresponding to amino acid residue 264 of SEQ ID NO: 2 may be A264G.

The substitution in the polypeptide chain at a position corresponding to amino acid residue 268 of SEQ ID NO: 2 may be a substitution with a polar, hydrophilic and/or neutral amino acid. Preferably, the substitution in the polypeptide chain at a position corresponding to amino acid residue 268 of SEQ ID NO: 2 may be a substitution that decreases the side chain volume of the residue at position 268. The hydroxyl in the side chain of residue 268 is crucial for catalysis, accordingly, the substitution in the polypeptide chain at a position corresponding to amino acid residue 268 of SEQ ID NO: 2 may be T268S.

The substitution in the polypeptide chain at a position corresponding to amino acid residue 328 of SEQ ID NO: 2 may be a substitution with an aliphatic and/or neutral amino acid. Preferably, the substitution in the polypeptide chain at a position corresponding to amino acid residue 328 of SEQ ID NO: 2 may be a substitution that decreases the side chain volume of the residue at position 328. The substitution in the polypeptide chain at a position corresponding to amino acid residue 328 of SEQ ID NO: 2 may be A328G.

The substitution in the polypeptide chain at a position corresponding to amino acid residue 354 of SEQ ID NO: 2 may be a substitution with an aromatic, hydrophobic and/or neutral amino acid. Preferably, the substitution in the polypeptide chain at a position corresponding to amino acid residue 354 of SEQ ID NO: 2 may be a substitution that increases the side chain volume of the residue at position 354. The substitution in the polypeptide chain at a position corresponding to amino acid residue 354 of SEQ ID NO: 2 may be M354F.

### Mutant CYP102A Enzymes

The invention also relates to a mutant CYP102A enzyme comprising a heme monooxygenase domain comprising a P450 fold, and said mutant CYP102A enzyme comprises substitution(s) in the polypeptide chain of a wild-type CYP102A enzyme at one or more positions corresponding to amino acid residue positions 435, 82, 184, 260 and/or 72 of SEQ ID NO: 2, thereby enhancing monooxygenase activity and/or altering product selectivity of the mutant enzyme. The enhanced monooxygenase may be enhanced monooxygenation of a secosteroid, preferably of a vitamin D and more preferably of a vitamin D₃ or vitamin D₂. The altered product selectivity may be the production of a secosteroid product oxidised at C25, preferably of a vitamin D oxidised at C25 and more preferably of a vitamin D₃ or vitamin D₂ oxidised at C25. The wild-type CYP102A enzyme does not act as a monooxygenase for secosteroid products.

The mutant CYP102A enzyme (for example when used in the process of the invention) may have a conversion rate of at least 10%, such as at least 15%, at least 20%, at least 25%, at least 30%, at least 35%, at least 40%, at least 45%, at least 50%, at least 55%, at least 60%, at least 65%, at least 70%, at least 75%, or at least 80% or more. The mutant CYP102A enzyme may have a turnover number of at least 50, such as at least 100, at least 200, at least 300, at least 400, at least 500, at least 600, at least 700 or at least 800 or more. The mutant CYP102A enzyme may have a conversion rate of at least 10% and a TON of at least 50.

The substitutions at one or more positions corresponding to amino acid positions 435, 82, 184, 260 and/or 72 of SEQ ID NO: 2 may be any of those discussed above in relation to the process of the invention. The mutant CYP102A enzyme may comprise one or more additional substitutions discussed above in relation to the process of the invention.

Also provided is a process for oxidising an organic compound substrate, comprising the step of contacting said organic compound substrate with the mutant CYP102A enzyme of the invention. The organic compound is typically any organic compound capable of being oxidized by a monooxygenase enzyme. The suitability of any organic compound for oxidation by a monooxygenase enzyme may be routinely determined by the methods described herein. The organic compound may be a steroid, preferably a secosteroid.

The oxidation process causes the formation of a C—O bond in the compound, generally as the alcohol from the oxidation of a carbon-hydrogen bond, but an epoxide may be formed from the oxidation of a C═C bond. The oxidation may thus introduce an alcohol, aldehyde, ketone or epoxide group. Alternatively the oxidation may cause the further oxidation of an oxygen containing group, such as converting an alcohol group into an aldehyde or ketone. 1, 2 or more carbon atoms may be attacked in the same substrate molecule. Oxidation can also result in N- and O-dealkylation of the substrate molecule.

The substrate can either be a natural substrate of a wild type CYP102A enzyme or a substrate which is not normally a substrate for the wild type enzyme, but which is capable of being utilized as such in the mutant enzyme. Examples of natural substrates for CYP102A enzymes are branched and straight chain fatty acids, which are hydroxylated by wild type CYP102A1 at sub-terminal positions (@-1 to @-3). Preferred examples are lauric acid, undecanoic acid, decanoic acid, nonanoic acid and octanoic acid. Examples of non-natural substrates of a wild type CYP102A enzyme that may be utilized with the mutant CYP102A enzyme include steroids, such as secosteroids.

### Other Products of the Invention

The invention also relates to a polynucleotide which comprises a sequence which encodes the mutant CYP102A enzyme of the invention.

The polynucleotide may be in the form of a vector. The vector is typically a transposon, plasmid, virus or phage vector. It typically comprises an origin of replication. It typically comprises one or more selectable marker genes, for example an ampicillin resistance gene in the case of a bacterial plasmid. The vector is typically introduced into host cells using conventional techniques including calcium phosphate precipitation, DEAE-dextran transfection, or electroporation.

The invention also relates to a cell that expresses the mutant CYP102A enzyme of the invention. The cell may be prokaryotic cell. The cell may be a eukaryotic cell. The cell may be a strain of bacteria such as Escherichia coli, Pseudomonas sp., Rhodococcus sp. or Bacillus sp. The cell may be a strain of yeast, such as Pichia sp.

The invention also relates to a transgenic animal or plant that comprises cells that express the mutant CYP102A enzyme of the invention. The transgenic animal may be a non-mammalian animal, such as a non-human animal. The animal or plant is transgenic for one or more polynucleotides encoding the mutant CYP102A enzyme. They may be homozygotic or heterozygotic for such polynucleotides, which are typically transiently introduced into the cells, or stably integrated. The plant or animal may be obtained by transforming an appropriate cell (e.g. embryo stem cell, callus or germ cell), fertilizing the cell if required, allowing the cell to develop into the animal or plant and breeding the animal or plant if required. The animal or plant may be obtained by sexual or asexual reproduction (e.g. cloning), propagation of an animal or plant of the invention or of the F1 organism (or any generation removed from the F1, or the chimera that develops from the transformed cell).

The cell is typically produced by introducing into a cell (i.e. transforming the cell with) the vector comprising a polynucleotide that encodes the mutant CYP102A enzyme of the invention. It is to be understood that due to the degeneracy of the nucleotide code, more than one polynucleotide can encode each of the mutant CYP102A enzymes. It is also to be understood that the nucleotide sequence may be engineered to exhibit a codon bias suitable for a particular cell or organism. The vector may integrate into the genome of the cell or remain extra-chromosomal. The cell may develop into the animal or plant. Typically the coding sequence of the polynucleotide is operably linked to a control sequence which is capable of providing for the expression of the coding sequence by the host cell. The control sequence is generally a promoter, typically of the cell in which the monooxygenase is expressed.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

The processes disclosed herein may be performed in the cell expressing the mutant CYP102A enzyme. The process may be performed in vitro, such as in culture, in vivo or in planta.

### Organic Compounds

Using the process of the invention, the inventors have also obtained a process to obtain two novel compounds 25,26-epoxy-vitamin D₃ [IUPAC name: (1S,Z)-3-(2-((1R,3aS,7aR,E)-7a-methyl-1-((2R)-5-(2-methyloxiran-2-yl) pentan-2-yl) octahydro-4H-inden-4-ylidene)ethylidene)-4-methylenecyclohexan-1-ol] and 25,28-dihydroxy-vitamin D₂ [IUPAC name: (3S,6R,E)-6-((1R,3aS,7aR,E)-4-((Z)-2-((S)-5-hydroxy-2-methylenecyclohexylidene)ethylidene)-7a-methyloctahydro-1H-inden-1-yl)-2-methylhept-4-ene-2,3-diol]. To the best of the inventors' knowledge, these compounds have not been previously generated. The inventors have arrived at these compounds using mutant CYP102A enzymes of the invention, as explained in Examples 2 and 4, respectively.

25,26-Epoxy-vitamin D₃ and 25,28-dihydroxy-vitamin D₂ are similar in structure to 25-hydroxy-vitamin D₃ and 25-hydroxy-vitamin D₂, respectively, and are thus likely substrates for oxidation by CYP27B1 into 1-hydroxy, 25,26-epoxy-vitamin D₃ and 1,25,28-trihydroxy-vitamin D₂. Due to high structural similarity with the natural ligands of the vitamin D receptor, both 1-hydroxy, 25,26-epoxy-vitamin D₃ and 1,25,28-trihydroxy-vitamin D₂ are likely also ligands of the vitamin D receptor.

25,26-Epoxy-vitamin D₃ and 25,28-dihydroxy-vitamin D₂ may be used as alternatives to 25-hydroxy-vitamin D₃ and 25-hydroxy-vitamin D₂ in pharmaceutical compositions, health supplements or animal feeds.

### Compositions

The process of the invention may comprise oxidizing vitamin D₃ to 25-hydroxy-vitamin D₃ or 1,25-dihydroxy-vitamin D₃ and formulating the 25-hydroxy-vitamin D₃ or 1,25-dihydroxy-vitamin D₃ in a pharmaceutical composition, a health supplement or an animal feed. The process of the invention may comprise oxidizing vitamin D₂ to 25-hydroxy-vitamin D₂ and formulating the 25-hydroxy-vitamin D₂ in a pharmaceutical composition, a health supplement or an animal feed.

Also disclosed herein is a pharmaceutical composition, a health supplement or an animal feed comprising the 25-hydroxy-vitamin D₃ or 1,25-dihydroxyvitamin D₃ or 25-hydroxy-vitamin D₂ produced by the method of the invention.

The pharmaceutical composition, a health supplement or an animal feed may comprise one or more additional agents. For example, the pharmaceutical composition may comprise a pharmaceutically acceptable excipient, carrier, diluent, buffer, stabiliser or other materials well known to those skilled in the art. Such materials should be non-toxic and should not interfere with the efficacy of the active ingredient. The pharmaceutical carrier or diluent may be, for example, an isotonic solution.

The precise nature of the carrier or other material may depend on the route of administration, e.g. oral, intravenous, cutaneous or subcutaneous, nasal, intramuscular and intraperitoneal routes. For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Oral formulations include such normally employed excipients as, for example, pharmaceutical grade mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to an individual may be provided with an enteric coating comprising, for example, Eudragit “S”, Eudragit “L”, cellulose acetate, cellulose acetate phthalate or hydroxypropylmethyl cellulose.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Administration may be in a “prophylactically effective amount” or a “therapeutically effective amount” (as the case may be, although prophylaxis may be considered therapy), this being sufficient to show benefit to the individual, e.g. an effective amount to prevent or delay onset of a disease or condition, to ameliorate one or more symptoms, to induce or prolong remission, or to delay relapse or recurrence.

Administration may not require a therapeutically effective amount. Administration may be in an amount to ensure the recommended daily allowance of vitamin D is reached. The individual may already have a source of vitamin D and thus may only need to an amount to reach the recommended daily allowance.

The dose may be determined according to various parameters, especially according to the substance used; the species; the age, weight and condition of the individual to be treated; the route of administration; and the required regimen. A physician will be able to determine the required route of administration and dosage for any particular individual. A typical daily dose is about 20 μg of vitamin D per day in humans. The Endocrine Society states, for example, that to maintain serum 25 (OH) D levels above 75 nmol/L (30 ng/ml), adults might need at least 37.5 to 50 μg (1,500-2,000 IU)/day of supplemental vitamin D, and children and adolescents might need at least 25 μg (1,000 IU)/day. The recommended daily allowance (RDA) for vitamin D is 600 IU (15 μg) per day for adults. However, some experts recommend that adults take even more-up to 1000-2000 IU (25-50 μg) per day. The vitamin D content of human milk is related to the mother's vitamin D status; studies suggest that the breastmilk of mothers who take daily supplements containing at least 50 μg (2,000 IU) vitamin D₃ have higher levels of the nutrient. 25-Hydroxy-vitamin D₃ at 20 μg per day has been shown to be a far more effective human supplement than the same dose of vitamin D₃ in raising serum 25-hydroxy-vitamin D₃ levels. The dose of vitamin D supplement may be provided as a single dose or may be provided as multiple doses, for example taken at regular intervals, for example 2, 3 or 4 doses administered hourly.

Vitamin D₃ is a critical component feeds in farming. It is present at ˜25 μg/kg in fish feed, ˜125 μg/kg in poultry feed, ˜250 μg/kg for cattle, and up to ˜750 μg/kg of feed for dairy cows. 25-Hydroxy-vitamin D₃ has been used as a more beneficial feed for poultry at ˜70 μg/kg of feed.

Examples of the techniques and protocols mentioned above can be found in Remington's Pharmaceutical Sciences, 20th Edition, 2000, pub. Lippincott, Williams & Wilkins.

### EXAMPLES

### Example 1. Design and Engineering of CYP102A1 for the Oxidation of Vitamin D₃

The wild-type CYP102A1 enzyme is inactive for vitamin D₃ oxidation. The inventors investigated whether the enzyme could be engineered to provide this activity.

Since vitamin D₃ is formed via opening of the B-ring of the steroidal core, CYP102A1 variants with steroid oxidation activity were chosen as starting point to search for vitamin D₃ oxidation activity. Steroid oxidation by engineered CYP102A1 was first reported over 15 years ago, when the oxidation of testosterone was investigated. Reports on CYP102A1 engineering by directed evolution, combinatorial active-site saturation test (CAST) and iterative saturation mutagenesis (ISM) for testosterone and progesterone oxidation followed. Rational engineering of CYP102A1 to oxidise steroids including androstenedione, testosterone and dehydroepiandrosterone using a library of CYP102A1 enzyme variants has been reported. However, there has been no report of CYP102A1 oxidation of steroids with long side chains at C17, such as cholesterol, which are more closely related to vitamin D₃.

A library of ˜100 CYP102A1 variants that showed high activity and varied product profile for steroid oxidation (Chen et al., ACS Catal. 2020, 10, 8334-8343) was screened for the oxidation of vitamin D₃. These variants were designed by scanning glycine mutagenesis of residues close to the heme; combinations of mutations such as F87A, I263G, A264G and A328G were found to promote steroid oxidation. In Chen et al, the 1263G mutation was found to play a crucial role and was the base mutation to which other mutations were added. Molecular dynamics simulations showed that it altered the conformation of the I helix, causing local unwinding of the helix in the vicinity of the mutation which created space to accommodate the increased size of the steroid. However, this mutation was not effective in promoting vitamin D₃ oxidation and none of the variants showed significant activity for the oxidation of vitamin D₃.

A different approach was thus required. In designing new variants that might show C25 oxidation of vitamin D₃, the inventors noted that the well-known mutation F87A was required for steroid oxidation activity; this was selected as a base mutation in the new variants. For C25 oxidation, vitamin D₃ had to bind within the active site pocket with the isopropyl end of the C17 side chain being closest to the oxygen atom of the ferryl intermediate. This was identified to be achievable by accommodating the A-ring deep inside the pocket, e.g., in the vicinity of the A82 side chain at the far end of the substrate pocket. Alternatively, this end of the pocket could be blocked off to force the vitamin D₃ substrate to bind with the C17 side chain being close to the heme iron. The inventors identified mutations including A82M as suitable. The side chain volume of residues close to the heme could be reduced to create space for vitamin D₃ binding. Screening results indicated that the I263G mutation from Chen et al was ineffective in promoting vitamin D₃ oxidation. This was because the 1263 side chain is high in the substrate pocket, while the other I helix residues T260 and A264 are close to the heme. The inventors substituted T260 and A264 with glycine, as well as substituting A328 with glycine, another residue in the immediate vicinity of the heme. The side chain alcohol group of Thr268 is crucial for oxygen binding and O—O bond cleavage to form the ferryl, Compound I species [(P^{•+})Fe^{IV}(═O)] which is the active intermediate for C—H bond oxidation. T268 was substituted with Ser, which maintained the hydroxyl group on the side chain to fulfil the role of oxygen binding and activation but the side-chain methyl group was removed which created space to accommodate a substrate. The inventors identified the substrate channel residues S72, A330 and S332 for substitution with residues with bulkier side chains to promote substrate binding close to the heme. The inventors included a number of variants, such as the 1263G mutation, as a control. The new library of CYP102A1 variants was generated and screened for vitamin D₃ oxidation, as shown in Example 2.

### Example 2. Initial Enzyme Library Screening and Characterisation of Vitamin D₃ Oxidation Products

HPLC analysis of organic extracts from in vitro screening scale (0.5 mL) reactions showed that 11 out the 48 new variants possessed >10% vitamin D₃ conversion activity (total turnover number ˜50, Table 1). Active variants were selected for preparative scale reactions, from which three oxidation products were isolated and characterised by their NMR and MS data as the target product 25-hydroxy-vitamin D₃ (1), the diol 23,25-dihydroxy-vitamin D₃ (2), and the epoxide 25,26-epoxy-vitamin D₃ (3). The diol 2 was mostly likely formed via further oxidation of 1 at C23. The epoxide 3 likely arose from initial desaturation across the C25-C26 bond to form the terminal alkene followed by epoxidation.

The product profiles (Table 1) revealed that combinations of mutations A82M, F87A, A184I, T260G and T268S promoted 25-hydroxylation of vitamin D₃ to give 1. Very high (>99%) selectivity was possible; the variants R19/F87A/T268S and R19/F87A/A82M/T260G/A328G/A184I/A330V (Entries 6 & 10, Table 1) gave >99% of 1 although the activities were low. The most active variants, such as R19/F87A/A82M/T260G/A184I/A328G (total turnover number for the formation of 1, TON=285), had lower but still significant (>80%) selectivity for 1 (Entry 5, Table 1). The R19/F87A/A82M/T260G/A184I variant gave 90% of 1 but with a lower activity (TON=180, Entry 4, Table 1).

TABLE 1
(FIRST GENERATION MUTANTS).

| ENTRY | CYP102A1 variant | 1 | 2 | 3 | Conv. | TON |
|---|---|---|---|---|---|---|
| 1 | R19 F87A A82M T260G | 73% | — | 3% | 30% | 110 |
| 2 | K19 F87A A82M T260G T268S | 43% | — | — | 14% | 30 |
| 3 | R19 F87A A82M T260G A328G | 50% | — | — | 24% | 60 |
| 4 | R19 F87A A82M T260G A184I | 90% | — | 8% | 40% | 180 |
| 5 | R19 F87A A82M T260G A184I A328G | 85% | 9% | 6% | 67% | 285 |
| 6 | R19 F87A A82M T260G A184I A328G A330V | >99% | — | — | 9% | 45 |
| 7 | R19 F87A A82M T260G A184I A328G A330I | 64% | 5% | — | 22% | 70 |
| 8 | R19 F87A A82M T260G A184I A328G A330L | 80% | 7% | — | 15% | 60 |
| 9 | R19 F87A A82M T260G A184I A328G S72F | 50% | — | — | 14% | 35 |
| 10 | R19 F87A T268S | >99% | — | — | 16% | 80 |
| 11 | R19 F87A T268S A328I | 76% | — | — | 29% | 110 |

Table 1. Activity and product selectivity (HPLC analysis) for the hydroxylation of cholecalciferol (vitamin D₃) catalysed by selected cytochrome P450_{BM3} variants. The substrate-to-enzyme concentration ratio was 500:1 (1 mM vitamin D₃ and 2 μM CYP102A1 enzyme). Conv. is the percentage of the vitamin D₃ substrate converted to products. TON is the turnover number of the variant for the formation of 25-hydroxy-vitamin D₃ (1). K19 = H171L/Q307H/N319Y; R19 = R47L/Y51F/K19.

The results showed that the F87A/A82M combination of mutations promoted vitamin D₃ oxidation by CYP102A1; the only active variants without this combination were those with the T268S mutation (Entries 10 and 11, Table 1). The I263G mutation was not effective, as indicated by the initial screening, nor was the I263G/A264G combination, e.g., the K19/F87A/A82M/1263G/A264G variant of the new library was inactive (data not shown). Mutations of A330 to residues with larger side chains lowered the activity as much as six-fold when introduced to the most active variant within this new library (Entries 6-8). Mutations of S72 to residues with larger side chains also lowered activity in the context of the first generation mutants (Entry 9). Variability in success of combining mutations to promote formation of the desired product was further illustrated by the lower activity of the K19/F87A/A82M/T260G/T268S variant that contained the activity-promoting mutations A82M and T268S. Similarly, while the R19/F87A/A82M/T260G/A184I variant oxidised vitamin D₃ (Entry 4), the K19/F87A/A82M/T260G/A184I variant was inactive. The difference between these variants was in the addition of the R47L/Y51F combination in the R19 base variant compared to K19.

It was concluded that the base mutations K19 and R19 interfered with the effects of the activity-enhancing mutations at A82, F87, T260G, etc. In designing a new generation of variants for vitamin D₃ oxidation, the K19 and R19 base variant mutations were removed. The most effective mutation combination of F87A/A82M/T260G was retained as the starting point, as was the A184I mutation which enhanced both the activity and selectivity.

### Example 3. Second Generation Variants

The F87A/A82M/A184I/T260G variant showed higher conversion than the analogue with the R19 set of mutations added (Table 2, Entry 22 vs. Table 1, entry 4). Therefore, screening reactions were conducted at a higher substrate-to-enzyme ratio of 1000:1. Under these conditions, the F87A/A82M/A184I/T260G variant showed 40% conversion with 75% selectivity and a turnover number of 300 for 25-(OH)-D₃ formation. The wild-type enzyme and the F87A variant did not show detectable activity. Interestingly, the F87A/A82M variant showed 28% conversion and 72% selectivity for 25-(OH)-D₃, and both the A184I and T206G mutations lowered the activity and selectivity when added to this F87A/A82M double mutation variant (Table 2, Entries 14 and 16-17) but there was an increase in activity and selectivity when all four mutations were combined (Entry 22). The results highlighted the important effect of combinations of mutations in raising activity and/or selectivity.

TABLE 2
Activity and product selectivity (HPLC analysis) for the hydroxylation of
cholecalciferol (vitamin D₃) with second generation CYP102A1 variants.
The substrate-to-enzyme concentration ratio was 1000:1 (2 mM vitamin
D₃, 2 μM CYP102Al enzyme). Conv. is the percentage of substrate
converted to products. TON is the turnover number of the variant for
the formation of 25-hydroxy-vitamin D₃ (1).

| Entry | CYP102A1 variant | 1 | Conv. | TON |
|---|---|---|---|---|
| 12 | Wild-type | — | — | — |
| 13 | F87A | — | — | — |
| 14 | F87A A82M | 72% | 28% | 200 |
| 15 | F87A A82M S72A | 65% | 46% | 300 |
| 16 | F87A A82M A184I | 41% | 21% | 85 |
| 17 | F87A A82M T260G | 38% | 15% | 60 |
| 18 | F87A A82M E435M | 67% | 66% | 440 |
| 19 | F87A A82M T260G S72A | 47% | 21% | 100 |
| 20 | F87A A82M T260G E435M | 73% | 52% | 380 |
| 21 | F87A A82M S72A A184I | 50% | 52% | 260 |
| 22 | F87A A82M A184I T260G | 75% | 40% | 300 |
| 23 | F87A A82M A1841 E435M | 61% | 53% | 320 |
| 24 | F87A A82M S72A E435M | 45% | 55% | 245 |
| 25 | F87A A82M S72A A184I T260G | 63% | 37% | 235 |
| 26 | F87A A82M A184I T260G E435M | 72% | 50% | 360 |
| 27 | F87A A82M A184I S72A E435M | 71% | 54% | 390 |
| 28 | F87A A82M S72A T260G E435M | 64% | 42% | 265 |
| 29 | F87A A82M A184I T260G S72A E435M | 73% | 79% | 570 |
| 30 | F87A A82M A184I T260G S72A E435M | 70% | 80% | 560 |
| | N239H | | | |
| 31 | F87A A82M A184I T260G S72A E435I | 75% | 83% | 620 |
| 32 | F87I A82M A184I T260G S72A E435M | 81% | 83% | 675 |
| 33 | F87V A82M A184I T260G S72A E435M | 49% | 77% | 380 |
| 34 | F87V A82M A184I T260G S72A E435I | 50% | 83% | 410 |
| 35 | F87A A82M A184I T260G S72A E435T | 70% | 77% | 540 |
| 36 | F87I A82M A184I T260G S72A E435I | 83% | 83% | 690 |
| 37 | F87I A82M A184I T260G S72A E435I | 82% | 83% | 685 |
| | N239H | | | |
| 38 | F87I A82M A184I T260G S72A E435M | 86% | 80% | 690 |
| | L29M | | | |

—: No detectable activity.

Vitamin D₃ was then computationally docked into the active site of molecular dynamics simulation structures of the F87A/A82M/A184I/T260G variant. From the substrate binding model, active site residues within 5 Å of the bound substrate were selected for substitutions with amino acid residues with smaller as well as larger side chains to probe the effects on vitamin D₃ oxidation. Mutations were introduced at P6, L19, L20, P25, V26, L29, K69, S72, A74, L75, V78, V178, L181, M185, L188, I259, A330, S332, M354, Q403, L437, and T438. We also targeted salt bridge and hydrogen bonding interactions that linked secondary structure elements, with the view to loosen the structure and introduce greater flexibility to enable the entry and binding of unnatural substrates such as vitamin D₃. To this end, hydrophobic substitutions were introduced at residues R79, N239, R255, E435 and K440.

Most of the introduced mutations at these target residues had minor effects on the activity of the F87A/A82M/A184I/T260G base variant or abolished the activity altogether, except the mutations S72A and E435M (Tables 2 and 3). The S72A mutation increased the D₃ oxidation activity between 50% and two-fold when added to various precursor variants (see, for example, Table 2, Entries 14&15; 17&19; 16&21). The E435M mutation significantly increased vitamin D₃ conversion from 37% to 79%, and the selectivity for C25 hydroxylation from 63% to 73% when added to the F87A/A82M/A184I/T260G/S72A variant, leading to a TON of 570 for the formation of 25-(OH)-VD₃ for the F87A/A82M/A184I/T260G/S72A/E435M variant (Entry 29). This activity enhancing effect of the E435M mutation was observed with other precursors (see, for example, Table 2, Entries 14&18, 17&20, 16&23, etc.). The carboxylate group of the E435 side-chain forms a hydrogen bond with the backbone carbonyl oxygen of V26. Disruption of this bonding interaction imparts greater flexibility to this part of the substrate access channel in P450_{BM3} which could promote vitamin D₃ oxidation. Substitutions of E435 with other hydrophobic residues (Ala, Val, Ile, Leu, Phe) showed that the E435I mutation was even more effective than the E435M, with a TON of 620 for 25-OH-D₃ formation via increased conversion and C25 selectivity for the F87A/A82M/A184I/T260G/S72A/E435I variant (Entry 31). The E435T mutation had been reported by Li et al. (Appl. Biochem. Biotechnol. 2008, 144, 27-36) to increase the indigo formation activity of the A74G/F87V/L188Q variant of CYP102A1 by indole oxidation, but hydrophobic substitutions were less effective, e.g., the activity of the best such variant with the E435F mutation was ˜60% of the activity of the E435T variant. The E435T mutation was found to be less effective than the E435I and E435M mutation in promoting vitamin D₃ oxidation (Entry 35).

Different hydrophobic substitutions were then introduced to F87. The F87I mutation increased the activity by ˜10% to a TON of ˜700 for the E435I and E435M variants (Table 2, Entries 32 & 36), while the F87V mutation lowered the C25 selectivity (Entries 33 & 34).

TABLE 3
Activity and product selectivity (HPLC analysis) for the oxidation of cholecalciferol
(vitamin D₃) catalysed by CYP102A1 variants. The substrate-to-enzyme concentration
ratio was 1000:1 (2 mM vitamin D₃, 2 μM CYP102Al enzyme). Conv. is the percentage
of substrate converted to products. TON is the turnover number of the variant for
the formation of 25-hydroxy-vitamin D₃, 1.

| CYP102A1 variant | Conv. | 1 | 2 | TON |
|---|---|---|---|---|
| WT | — | — | — | — |
| F87A | — | — | — | — |
| F87A A82M | 28% | 72% | <5% | 200 |
| F87A A184I S72A T260G E435M | 24% | 64% | 7% | 155 |
| F87A A184I S72A T260G E435M A82M | 79% | 73% | 15% | 570 |
| F87A A184I S72A T260G E435T | 11% | 34% | 7% | 35 |
| F87A A184I S72A T260G E435T A82M | 77% | 70% | 6% | 540 |
| F87A A82M A184I S72A T260G | 37% | 63% | 6% | 235 |
| F87A A82M A184I S72A T260A | 15% | 38% | 5% | 55 |
| F87A A82M A184I S72A T260G L181F | 11% | 17% | 6% | 20 |
| F87A A82M A184I S72A E435M T260G | 79% | 73% | 15% | 570 |
| F87A A82M A184I S72A E435M T260A | 20% | 48% | 9% | 95 |
| F87A A82M A184F S72A T260G E435M | 42% | 72% | 5% | 300 |
| F87A A82M A184M S72A T260G E435M | 29% | 64% | 7% | 185 |
| F87A A82M A184I S72A T260G E435I | 83% | 75% | 14% | 620 |
| F87A A82M A184M S72A T260G E435I | 12% | 29% | 9% | 35 |
| F87A A82M A184V S72A T260G E435I | 34% | 67% | 5% | 230 |
| F87A A82M A184L S72A T260G E435I | 35% | 69% | 5% | 245 |
| F87A A82M A184I S72A T260G E435I V26L | 14% | 53% | 5% | 75 |
| F87A A82M A184I S72A T260G E435I V26M | 23% | 67% | 5% | 160 |
| F87A A82M A184I S72A T260G E435I V26H | 10% | 30% | 9% | 30 |
| F87A A82M A184I S72A T260G E435M V26L | 46% | 74% | 5% | 340 |
| F87A A82M A184I S72A T260G E435M V26M | 45% | 76% | 5% | 345 |
| F87A A82M A184I S72A T260G E435M V26H | 47% | 76% | 5% | 360 |
| F87A A82M A184I S72A T260G E435T V26L | 37% | 9% | <5% | 35 |
| F87A A82M A184I S72A T260G E435T V26M | 10% | 18% | 10% | 20 |
| F87A A82M A184I S72A T260G E435T V26H | 9% | 17% | 6% | 15 |
| F87I A82M A184I T260G E435M S72A | 83% | 81% | <5% | 675 |
| F87I A82M A184I T260G E435M S72V | 46% | 81% | <5% | 375 |
| F87I A82M A184I T260G E435M S72W | 11% | 34% | <5% | 40 |
| F87I A82M A184I T260G E435I S72A | 83% | 83% | <5% | 690 |
| F87I A82M A184I T260G E435I S72V | 54% | 85% | <5% | 460 |
| F87I A82M A184I T260G E435I S72W | 20% | 62% | <5% | 130 |
| F87A A82M A184I T260G E435M | 50% | 72% | 5% | 360 |
| F87A A82M A184I T260G E435M K69I | 17% | 15% | 8% | 25 |
| F87A A82M A184I T260G E435M K69R | 26% | 20% | 11% | 50 |
| F87A A82M T260G E435M | 52% | 73% | 6% | 380 |
| F87A A82M T260G E435M V178F | 31% | 51% | 5% | 160 |
| F87A A82M T260G E435M V178W | 29% | 51% | 5% | 150 |
| F87I A82M S72A T260G E435M A184I | 83% | 81% | <5% | 675 |
| F87I A82M S72A T260G E435M V178F | 55% | 75% | 6% | 410 |
| F87I A82M S72A T260G E435M V178W | 75% | 80% | 8% | 605 |
| F87I A82M S72A T260G E435I A184I | 83% | 83% | 8% | 690 |
| F87I A82M S72A T260G E435I V178F | 43% | 70% | <5% | 300 |
| F87I A82M S72A T260G E435I V178W | 59% | 80% | <5% | 475 |
| F87I A82M S72A T260G E435M A184I M354F | 79% | 85% | <5% | 670 |
| F87I A82M S72A T260G E435I A184I M354F | 14% | 13% | 7% | 20 |
| F87A A82M A184I T260G E435M M354L | 71% | 76% | <5% | 540 |
| F87A A82M A184I S72A T260G E435M Y51F | 67% | 76% | 13% | 510 |
| F87A A82M A184I S72A T260G E435M H171L | 10% | 9% | <5% | 10 |
| F87A A82M A184I S72A T260G E435M Q307H | 11% | 15% | 6% | 20 |
| F87A A82M A184I S72A T260G E435M N319Y | 61% | 75% | 11% | 460 |
| F87A A82M A184I S72A T260G E435M Q403P | 11% | 23% | 10% | 25 |
| F87A A82M A184I S72A T260G E435M V26L H171L | 12% | 16% | 6% | 20 |
| F87A A82M A184I S72A T260G E435M H171L L181F | 12% | 15% | 5% | 20 |
| F87A A82M A184I S72A T260G E435M V26L N239H | 68% | 77% | 8% | 530 |
| F87A A82M A184I S72A T260G E435M H171L N239H | 12% | 16% | 6% | 20 |
| F87A A82M A184I S72A T260G E435M L181F N239H | 32% | 43% | 29% | 140 |
| F87A A82M A184I S72A T260G | 37% | 63% | 6% | 235 |
| F87A A82M A184I S72A T260G Y51F H171L | 10% | 14% | 7% | 15 |
| F87A A82M A184I S72A T260G Y51F Q403P | 10% | 14% | 8% | 15 |
| F87A A82M A184I S72A T260G V26L Y51F | 47% | 69% | 8% | 325 |
| F87A A82M A184I S72A T260G H171L Q403P | 9% | 14% | 6% | 15 |
| F87A A82M A184I S72A T260G V26L Q403P | 8% | 9% | <5% | 10 |
| F87A A82M A184I S72A T260G V26L H171L | 11% | 15% | <5% | 15 |
| F87A A82M A184I S72A T260G E435L | 29% | 65% | 5% | 190 |
| F87A A82M A184I S72A T260G E435Y | 25% | 57% | 7% | 140 |
| F87A A82M A184I S72A T260G E435W | 24% | 57% | 5% | 140 |
| F87A A82M A184I S72A T260G E435N | 17% | 42% | 9% | 70 |
| F87A A82M A184I S72A T260G E435Q | 49% | 69% | 12% | 340 |
| F87A A82M A184I S72A T260G E435R | 17% | 49% | 8% | 85 |
| F87A A82M A184I S72A T260G E435H | 45% | 66% | 18% | 300 |
| F87A A82M A184I S72A T260G E435D | 35% | 69% | 7% | 240 |
| F87A A82M A184I S72A T260G R19 | 69% | 66% | 23% | 455 |
| F87A A82M A184I T260G E435M | 50% | 72% | 5% | 360 |
| F87A A82M A184I T260G E435M R19 | 24% | 61% | 5% | 145 |
| F87A A82M A184I T260G E435I R19 | 8% | 16% | 6% | 15 |
| F87A A82M A184I T260G E435T R19 | 42% | 64% | 6% | 270 |
| F87V A82M A184I S72A T260G E435M | 77% | 49% | 40% | 380 |
| F87V A82M A184I S72A T260G E435M N239H | 71% | 50% | 41% | 360 |
| F87V A82M A184I S72A T260G E435I | 83% | 50% | 42% | 410 |
| F87V A82M A184I S72A T260G E435I N239H | 10% | 17% | 9% | 20 |
| F87I A82M A184I S72A T260G E435M | 83% | 81% | 10% | 675 |
| F87I A82M A184I S72A T260G E435M N239H | 79% | 75% | 13% | 590 |
| F87I A82M A184I S72A T260G E435M L29A | 68% | 82% | — | 555 |
| F87I A82M A184I S72A T260G E435M L29M | 80% | 86% | <5% | 690 |
| F87I A82M A184I S72A T260G E435M L75S | 14% | 14% | 6% | 20 |
| F87I A82M A184I S72A T260G E435M L75T | 15% | 13% | 6% | 20 |
| F87I A82M A184I S72A T260G E435M L188S | 54% | 76% | <5% | 410 |
| F87A A82M A184I S72A T260G E435M | 79% | 73% | 15% | 570 |
| F87S A82M A184I S72A T260G E435M | 70% | 76% | 7% | 530 |
| F87T A82M A184I S72A T260G E435M | 76% | 74% | 14% | 560 |
| F87A A82M A184I S72A T260G E435I | 83% | 75% | 14% | 620 |
| F87T A82M A184I S72A T260G E435I | 70% | 78% | 9% | 550 |
| F87I A82M A184I S72A T260G E435I | 83% | 83% | — | 690 |
| F87I A82M A184I S72A T260G E435I L75S | 71% | 84% | — | 600 |
| F871 A82M A184I S72A T260G E435I M185T | 61% | 79% | — | 480 |

—: No detectable activity.

### Example 4. The Oxidation of Vitamin D₂

Vitamin D₂ and D₃ differ by the presence of the C22-C23 double bond and the C24 methyl group in vitamin D₂. The rigidity of the double bond and altered steric demand introduced by the methyl group can affect P450 substrate recognition, as demonstrated by the oxidation of vitamin D₂ to its circulating form, 25-hydroxy-vitamin D₂, by a different liver P450 enzyme (CYP2R1) from that for vitamin D₃ (CYP27A1). Likewise, CYP2R1 does not oxidise vitamin D₃. It was therefore investigated whether a CYP102A1 variant capable of C25 oxidation of vitamin D₃ may also have vitamin D₂ oxidation activity.

TABLE 4
Activity and product selectivity (HPLC analysis) for the hydroxylation
of calciferol (vitamin D₂) with second generation CYP102A1 variants.
The substrate-to-enzyme concentration ratio was 1000:1 (2 mM vitamin
D₂, 2 μM CYP 102Al enzyme). Conv. is the percentage of substrate
converted to products. TON is the turnover number of the variant
for the formation of 25-hydroxy-vitamin D₂ (4).

| ENTRY | CYP102A1 variant | 4 | Conv. | TON |
|---|---|---|---|---|
| 39 | Wild-type | — | — | — |
| 40 | F87A | — | — | — |
| 41 | F87A A82M | 57% | 19% | 110 |
| 42 | F87A A82M A184I | 55% | 10% | 55 |
| 43 | F87A A82M S72A | 79% | 25% | 195 |
| 44 | F87A A82M T260G | 59% | 4% | 25 |
| 45 | F87A A82M E435M | 92% | 57% | 520 |
| 46 | F87A A82M T260G S72A | 45% | 16% | 70 |
| 47 | F87A A82M A184I T260G | 62% | 17% | 110 |
| 48 | F87A A82M A184I T260G R19 | 85% | 68% | 580 |
| 49 | F87A A82M A184I S72A | 23% | 70% | 160 |
| 50 | F87A A82M S72A E435M | 72% | 55% | 400 |
| 51 | F87A A82M A1841 E435M | 82% | 37% | 305 |
| 52 | F87A A82M T260G E435M | 95% | 28% | 270 |
| 53 | F87A A82M A184I T260G S72A | 59% | 31% | 190 |
| 54 | F87A A82M A184I S72A E435M | 93% | 38% | 355 |
| 55 | F87A A82M A184I T260G E435M | 94% | 35% | 330 |
| 56 | F87A A82M T260G S72A E435M | 93% | 47% | 440 |
| 57 | F87A A82M A184I T260G S72A E435T | 94% | 59% | 550 |
| 58 | F87A A82M A184I T260G S72A E435M | 96% | 71% | 685 |
| 59 | F87A A82M A184I T260G S72A E435I | 96% | 77% | 750 |
| 60 | F871 A82M A184I T260G S72A E435M | 88% | 81% | 720 |
| 61 | F871 A82M A184I T260G S72A E435I | 92% | 88% | 810 |
| 62 | F87V A82M A184I T260G S72A E435I | 50% | 80% | 400 |
| 63 | F87A A82M A184I T260G S72A E435M | 94% | 73% | 690 |
| | N239H | | | |
| 64 | F871 A82M A184I T260G S72A E435I | 88% | 89% | 785 |
| | N239H | | | |

—: No detectable activity.

Wild-type CYP102A1, the F87A variant and second-generation variants were tested for vitamin D₂ oxidation. The wild-type and F87A variant did not show vitamin D₂ oxidation activity. The second-generation variants were active, albeit at lower levels compared to vitamin D₃ (Table 4). The major product purified from a preparative scale reaction with the F87A/A82M/A184I/T260G/E435M variant was identified by its NMR and MS data as 25-hydroxy-vitamin D₂, 4 (94% selectivity, TON=330, Table 4, Entry 55). Also obtained by some variants was 25,28-dihydroxy-vitamin D₂. The activity and selectivity trends paralleled those for vitamin D₃ (Tables 4 and 5); thus, relatively low activity was observed with the F87A/A82M variant; the activity and selectivity for C25 oxidation increased with the introduction of the A184I/T260G couplet and further increases were observed as the S72A, E435M and E435I mutations were added. The most active and selective variant for vitamin D₃ oxidation, F87I/A82M/A184I/T260G/S72A/E435I, was also the most active and selective for 25-hydroxylation of vitamin D₂ (88% conversion, 92% selectivity, TON=810, Table 4, Entry 61).

TABLE 5
Activity and product selectivity (HPLC analysis) for the oxidation of calciferol
(vitamin D₂) catalysed by CYP102A1 variants. The substrate-to-enzyme concentration
ratio was 1000:1 (2 mM vitamin D₂, 2 μM CYP102Al enzyme). Conv. is the percentage
of substrate converted to products. TON is the turnover number of the variant for
the formation of 25-hydroxy-vitamin D₂, 4.

| CYP102A1 variant | Conv. | 4 | 5 | TON |
|---|---|---|---|---|
| WT | — | — | — | — |
| F87A | — | — | — | — |
| F87A A82M | 19% | 57% | 10% | 110 |
| F87A A184I S72A T260G E435M | 16% | 76% | <5% | 122 |
| F87A A184I S72A T260G E435M A82M | 71% | 96% | <4% | 685 |
| F87A A184I S72A T260G E435T | 6% | 25% | 8% | 15 |
| F87A A184I S72A T260G E435T A82M | 59% | 94% | — | 550 |
| F87A A82M A184I S72A T260G | 31% | 59% | 6% | 190 |
| F87A A82M A184I S72A T260A | 8% | 50% | — | 40 |
| F87A A82M A184I S72A T260G L181F | <5% | 11% | 7% | <10 |
| F87A A82M A184I S72A E435M T260G | 71% | 96% | <4% | 685 |
| F87A A82M A184I S72A E435M T260A | 21% | 82% | — | 175 |
| F87A A82M A184F S72A T260G E435M | 30% | 89% | — | 270 |
| F87A A82M A184M S72A T260G E435M | 22% | 86% | <5% | 190 |
| F87A A82M A184I S72A T260G E435I | 77% | 96% | <4% | 750 |
| F87A A82M A184M S72A T260G E435I | 22% | 86% | <5% | 190 |
| F87A A82M A184V S72A T260G E435I | 26% | 88% | — | 230 |
| F87A A82M A184L S72A T260G E435I | 24% | 87% | — | 215 |
| F87A A82M A184I S72A T260G E435I V26L | 5% | 38% | 6% | 20 |
| F87A A82M A184I S72A T260G E435I V26M | 13% | 77% | <5% | 100 |
| F87A A82M A184I S72A T260G E435I V26H | <5%% | 25% | — | 10 |
| F87A A82M A184I S72A T260G E435M V26L | 20% | 86% | — | 170 |
| F87A A82M A184I S72A T260G E435M V26M | 34% | 90% | — | 310 |
| F87A A82M A184I S72A T260G E435M V26H | 32% | 88% | — | 285 |
| F87A A82M A184I S72A T260G E435T V26L | 5% | 29% | 7% | 15 |
| F87A A82M A184I S72A T260G E435T V26M | <4% | — | — | <10 |
| F87A A82M A184I S72A T260G E435T V26H | <5% | — | — | <10 |
| F87I A82M A184I T260G E435M S72A | 81% | 88% | 11% | 720 |
| F87I A82M A184I T260G E435M S72V | 38% | 89% | — | 340 |
| F87I A82M A184I T260G E435M S72W | 12% | 69% | <5% | 85 |
| F87I A82M A184I T260G E435I S72A | 88% | 92% | <5% | 810 |
| F87I A82M A184I T260G E435I S72V | 41% | 91% | — | 375 |
| F87I A82M A184I T260G E435I S72W | 12% | 69% | — | 85 |
| F87A A82M A184I T260G E435M | 35% | 94% | — | 330 |
| F87A A82M A184I T260G E435M K69I | 7% | <5% | <5% | <10 |
| F87A A82M A184I T260G E435M K69R | 6% | 14% | <5% | <10 |
| F87A A82M T260G E435M | 28% | 95% | <5% | 270 |
| F87A A82M T260G E435M V178F | 18% | 70% | — | 120 |
| F87A A82M T260G E435M V178W | 12% | 54% | — | 70 |
| F87I A82M S72A T260G E435M A184I | 81% | 88% | 11% | 720 |
| F87I A82M S72A T260G E435M V178F | 72% | 86% | 5% | 620 |
| F87I A82M S72A T260G E435M V178W | 51% | 90% | — | 470 |
| F87I A82M S72A T260G E435I A184I | 88% | 92% | <5%% | 810 |
| F87I A82M S72A T260G E435I V178W | 58% | 92% | — | 535 |
| F87I A82M S72A T260G E435I V178F | 39% | 87% | — | 340 |
| F87I A82M S72A T260G E435M A184I M354F | 77% | 91% | — | 700 |
| F87I A82M S72A T260G E435I A184I M354F | 5% | 5% | — | <10 |
| F87A A82M A184I T260G E435M | 35% | 94% | — | 330 |
| F87A A82M A184I T260G E435M M354L | 36% | 86% | — | 315 |
| F87A A82M A184I S72A T260G E435M Y51F | 57% | 93% | <5% | 525 |
| F87A A82M A184I S72A T260G E435M H171L | 5% | 17% | — | <10 |
| F87A A82M A184I S72A T260G E435M Q307H | <5% | — | — | <10 |
| F87A A82M A184I S72A T260G E435M N319Y | 56% | 93% | — | 520 |
| F87A A82M A184I S72A T260G E435M Q403P | <5% | 29% | — | <10 |
| F87A A82M A184I S72A T260G E435M V26L H171L | 6% | 23% | — | 15 |
| F87A A82M A184I S72A T260G E435M H171L L181F | 5% | 12% | <5% | <10 |
| F87A A82M A184I S72A T260G E435M V26L N239H | 45% | 88% | <5% | 400 |
| F87A A82M A184I S72A T260G E435M H171L N239H | 5% | 6% | 5% | <10 |
| F87A A82M A184I S72A T260G E435M L181F N239H | 16% | 64% | 14% | 100 |
| F87A A82M A184I S72A T260G | 31% | 59% | 6% | 190 |
| F87A A82M A184I S72A T260G Y51F H171L | <5% | — | — | <10 |
| F87A A82M A184I S72A T260G Y51F Q403P | <5% | — | — | <10 |
| F87A A82M A184I S72A T260G V26L Y51F | 36% | 89% | <5% | 320 |
| F87A A82M A184I S72A T260G H171L Q403P | >5% | — | — | <10 |
| F87A A82M A184I S72A T260G V26L Q403P | <5% | — | — | <10 |
| F87A A82M A184I S72A T260G V26L H171L | <5% | — | — | <10 |
| F87A A82M A184I S72A T260G E435L | 15% | 82% | <5% | 125 |
| F87A A82M A184I S72A T260G E435Y | 14% | 76% | — | 105 |
| F87A A82M A184I S72A T260G E435W | 20% | 81% | — | 160 |
| F87A A82M A184I S72A T260G E435T | 59% | 94% | — | 550 |
| F87A A82M A184I S72A T260G E435N | 8% | 50% | — | 40 |
| F87A A82M A184I S72A T260G E435Q | 33% | 87% | — | 290 |
| F87A A82M A184I S72A T260G E435R | 7% | 51% | <5% | 40 |
| F87A A82M A184I S72A T260G E435H | 31% | 86% | <5% | 265 |
| F87A A82M A184I S72A T260G E435D | 34% | 88% | — | 300 |
| F87A A82M A184I S72A T260G | 31% | 59% | 6% | 190 |
| F87A A82M A184I S72A T260G R19 | 58% | 87% | 6% | 505 |
| F87A A82M A184I T260G E435M | 35% | 94% | — | 330 |
| F87A A82M A184I T260G E435M R19 | 17% | 75% | 6% | 130 |
| F87A A82M A184I T260G E435I R19 | <5% | — | — | <10 |
| F87A A82M A184I T260G E435T R19 | 40% | 72% | 16% | 290 |
| F87V A82M A184I S72A T260G E435M | 75% | 53% | 23% | 395 |
| F87V A82M A184I S72A T260G E435M N239H | 68% | 44% | 28% | 300 |
| F87V A82M A184I S72A T260G E435I | 80% | 50% | 46% | 400 |
| F87V A82M A184I S72A T260G E435I N239H | <5% | — | — | <10 |
| F87I A82M A184I S72A T260G E435M | 81% | 88% | 11% | 720 |
| F87I A82M A184I S72A T260G E435M N239H | 78% | 79% | 14% | 620 |
| F87I A82M A184I S72A T260G E435M L29A | 37% | 90% | — | 340 |
| F87I A82M A184I S72A T260G E435M L29M | 78% | 94% | <5% | 740 |
| F871 A82M A184I S72A T260G E435M L75S | 8% | <5% | — | <10 |
| F87I A82M A184I S72A T260G E435M L75T | 8% | <5% | — | <10 |
| F87I A82M A184I S72A T260G E435M L188S | 44% | 86% | <5% | 380 |
| F87A A82M A184I S72A T260G E435M | 71% | 96% | <4% | 685 |
| F87S A82M A184I S72A T260G E435M | 48% | 89% | — | 430 |
| F87T A82M A184I S72A T260G E435M | 70% | 91% | — | 630 |
| F87A A82M A184I S72A T260G E435I | 77% | 96% | <4% | 750 |
| F87T A82M A184I S72A T260G E435I | 75% | 92% | <5% | 695 |
| F87I A82M S72A T260G E435I A184I | 88% | 92% | <5% | 810 |
| F87I A82M A184I S72A T260G E435I L75S | 58% | 89% | <5% | 515 |
| F87I A82M A184I S72A T260G E435I M185T | 55% | 91% | — | 500 |

—: No detectable activity.

### Example 5. Materials and Methods

Screening of Vitamin D₃ and Vitamin D₂ for Oxidation by CYP102A1 Variants.

Both vitamin D₃ and D₂ were dissolved in EtOH and added as a 100 mM stock solution. Screening scale reactions were carried out in 200 mM phosphate buffer (pH 7.9) in a volume of 0.5 mL in 24-well plates. The final concentration of CYP102A1 variants was 2 μM; vitamin D₃ was at 1 mM (500:1) or 2 mM (1000:1) and vitamin D₂ at 2 mM (1000:1). Methyl-β-cyclodextrin (10 mM) was added to increase the solubility of vitamin D₃ in aqueous buffer. GDH (20 U/mL) and glucose (100 mM) were used to regenerate the NADPH cofactor. NADP⁺ (40 μM) was added to initiate the reaction. Screening plates were shaken in the dark at 120 rpm for 72 h at 20° C. and then extracted with 300 μL of ethyl acetate. After centrifugation at 14300 g to separate the phases, the organic extracts were analysed by reverse-phase HPLC on a C18 column (4.6 mm×10 cm; 5 μm), eluting with 95% acetonitrile in water for 20 mins at a flow rate of 1 mL/min. The retention times were: 23,25-dihydroxy-vitamin D₃ (2), 1.50 min; 25-hydroxy-vitamin D₃ (1), 2.27 min; 25,26-epoxy-vitamin D₃ (3), 3.10 min; vitamin D₃, 8.35 min; 25-hydroxy-vitamin D₂ (5), 2.51 min; vitamin D₂, 8.37 min.

### Preparative Scale Oxidation of Vitamin D₃.

The scalability of selective 25-hydroxylation of vitamin D₃ by engineered CYP102A1 in vitro was illustrated by the reaction catalysed by the R19/F87A/A82M/A184I/T260G variant. The reaction was scaled to 500 mL using the same concentrations of each component as for screening scale reactions except the substrate concentration which was raised to 3 mM (600 mg, 1.2 g/L) and 6 μM enzyme was used. The reaction mixture stirred at ambient temperature in the dark for 72 h. Aliquots were removed periodically to monitor the progress of the reaction by GC. A second aliquot of enzyme (6 μM) was added after 36 hours. After conversion reached >90% after 72 h, the reaction mixture was extracted three times with an equal volume of ethyl acetate. The combined extracts were washed with brine, dried with Na₂(SO₄) and the solvent was removed by rotary evaporation. The crude mixture was purified by silica gel column chromatography; the remaining vitamin D₃ substrate was eluted with a 5:1 mixture of petroleum ether (b.p. 30-40° C.) and ethyl acetate (petrol/EtOAc); the epoxide (3) was eluted with a 3:1 mixture of petrol/EtOAc; 25-hydroxy-vitamin D₃ (1) was eluted with 1:1 petrol/EtOAc, and the diol 2 was eluted with pure EtOAc. 25-(OH)-D₃ was isolated in 72% yield (400 mg) based on the amount of vitamin D₃ converted.

### Further Embodiments

Further embodiments of the invention are set out below:

1. A process for oxidising a secosteroid, comprising the step of contacting said secosteroid with a mutant CYP102A (Cytochrome P450 family 102A sub-family member) enzyme, wherein said CYP102A enzyme comprises a heme monooxygenase domain comprising a P450 fold, and said mutant CYP102A enzyme comprises substitution(s) in the polypeptide chain of a wild-type CYP102A enzyme at one or more positions, thereby enhancing monooxygenase activity and/or altering product selectivity of the mutant enzyme.

2. The process according to embodiment 1, wherein said mutant CYP102A enzyme comprises substitution(s) in the polypeptide chain of a wild-type CYP102A enzyme at one or more positions corresponding to amino acid residue positions 435, 82, 184, 260 and/or 72 of SEQ ID NO: 2.

3. The process according to embodiment 2, wherein said mutant CYP102A enzyme comprises one or more substitutions selected from E435I, E435M, E435T, A82M, A82L, A82I, A82F, A82W, A184I, T260G, T260A, S72A and/or S72G in SEQ ID NO:2, or corresponding substitutions in the polypeptide chain of a wild-type CYP102A enzyme at one or more positions corresponding to amino acid residues 435, 82, 184, 260 and/or 72 of SEQ ID NO: 2.

4 The process according to embodiment 2 or 3, wherein said mutant CYP102A enzyme additionally comprises one or more substitutions at one or more positions corresponding to amino acid residue positions 29, 78, 87, 178, 263, 264, 268, 328 and/or 354 of SEQ ID NO:2.

5. The process according to embodiment 4, wherein said mutant CYP102A enzyme comprises one or more substitutions selected from L29M, V78F, F87A, F87V, F87I, F87S, F87T, V178F, V178W, V178L, V178I, I263G, A264G, T268S, A328G, and/or M354F or corresponding substitutions at one or more positions corresponding to amino acid residue positions 29, 78, 87, 178, 263, 264, 268, 328 and/or 354 of SEQ ID NO: 2.

6. The process according to embodiment 4 or 5, wherein said mutant CYP102A enzyme comprises a said substitution at position 82 of SEQ ID NO:2 or a position corresponding thereto and a said substitution at position 87 of SEQ ID NO:2 or a position corresponding thereto.

7. The process according to embodiment 6, wherein said mutant CYP102A enzyme additionally comprises a said substitution at position 184 of SEQ ID NO:2 or a position corresponding thereto, a said substitution at position 260 of SEQ ID NO:2 or a position corresponding thereto, a said substitution at position 72 of SEQ ID NO:2 or a position corresponding thereto, and/or a said substitution at position 435 of SEQ ID NO:2 or a position corresponding thereto.

8. The process according to embodiment 7, wherein said mutant CYP102A enzyme comprises a said substitution at position 82 of SEQ ID NO:2 or a position corresponding thereto, a said substitution at position 87 of SEQ ID NO:2 or a position corresponding thereto and a said substitution at position 435 of SEQ ID NO:2 or a position corresponding thereto. 9. The process according to any one of the preceding embodiments, wherein said mutant CYP102A enzyme comprises one of the following groups of substitutions in SEQ ID NO:2:
a. A82M/F87A;
b. A82M/T260G;
c. A82M/A184I;
d. A82M/S72A;
e. A82M/E435I, E435M or E435T;
f. F87A/T260G;
g. F87A/A184I;
h F87A/S72A;
i. A82M/F87A/T260G;
j. A82M/F87A/A184I;
k A82M/F87A/E435I, E435M or E435T;
1. F87A/A184I/T260G;
m. F87A/A184I/S72A;
n. F87A/A184I/E435I, E435M or E435T;
o. F87A/A82M/A184I/T260G;
p. F87A/A82M/T260G/S72A;
q. F87A/A82M/A184I/T260G/S72A;
r. F87A, F87I or F87V/A82M/A184I/T260G/E435I, E435M or E435T;
S. F87A, F87I, F87V, F87S or F87T/A82M/A184I/T260G/S72A/E435I, E435M or E435T; or
t. F87I/A82M/A184I/T260G/S72A/E435M/L29M;
or corresponding substitutions in the polypeptide chain of a wild-type CYP102A enzyme at one or more positions corresponding to the amino acid residue positions of SEQ ID NO: 2 as listed in a.-t.

10. The process according to any one of the preceding embodiments, wherein said mutant CYP102A enzyme additionally comprises one or more substitutions at one or more positions corresponding to amino acid residue positions 47, 51, 74, 171, 188, 239, 259, 307, 319, 330 and/or 353 of SEQ ID NO:2, optionally selected from R47L, Y51F, A74G, H171L, N239H, I259V, L188Q, Q307H, N319Y, A330V, A330I, A330L, A330W and/or L353I in SEQ ID NO: 2, or corresponding substitutions in the polypeptide chain of a wild-type CYP102A enzyme at one or more positions corresponding to amino acid residue positions 47, 51, 74, 171, 188, 239, 259, 307, 319, 330 and/or 353 of SEQ ID NO: 2.

11. The process according to any one of the preceding embodiments, wherein the mutant CYP102A enzyme comprises a fusion of a heme monooxygenase domain to a reductase domain.

12. The process according to any one of the preceding embodiments, which is for oxidizing vitamin D₃.

13. The process according to embodiment 12, which is for producing 25-hydroxy-vitamin D₃.

14. The process of embodiment 13, further comprising oxidation of 25-hydroxy-vitamin D₃ to 1,25-dihydroxyvitamin D₃.

15. The process according to any one of embodiments 1-11, which is for oxidizing vitamin D₂.

16. The process according to embodiment 15, which is for producing 25-hydroxy-vitamin D₂.

17. The process according to any one of the preceding embodiments, wherein the mutant CYP102A enzyme is a mutant CYP102A1 enzyme.

18. A mutant CYP102A (Cytochrome P450 family 102A sub-family member) enzyme, wherein said CYP102A enzyme comprises a heme monooxygenase domain comprising a P450 fold, and said mutant CYP102A enzyme comprises substitution(s) in the polypeptide chain of a wild-type CYP102A enzyme at one or more positions corresponding to amino acid residue positions 435, 82, 184, 260 and/or 72 of SEQ ID NO: 2, thereby enhancing monooxygenase activity and/or altering product selectivity of the mutant enzyme.

19. The mutant CYP102A enzyme according to embodiment 18, which is as defined in any one of embodiments 3 to 11 and 17.

20. A polynucleotide which comprises a sequence which encodes an enzyme as defined in embodiment 18 or 19, optionally in the form of a vector.

21. A cell which expresses an enzyme as defined in embodiment 18 or 19.

22. The cell according to embodiment 21 which is a prokaryotic or eukaryotic cell.

23. The cell according to embodiment 22, which is a strain of Escherichia coli, Pseudomonas sp., yeast, Pichia sp., Rhodococcus sp., Bacillus sp.

24. A transgenic animal or plant whose cells are as defined in any one of embodiments 21 to 23.

25. The process according to any one of embodiments 1 to 17, wherein the secosteroid is oxidized in a cell according to any one of embodiments 21 to 23.

26 The process according to any one of embodiments 1 to 14, 17 and 25, comprising oxidizing vitamin D₃ to 25-hydroxy-vitamin D₃ or 1,25-dihydroxy-vitamin D₃ and formulating the 25-hydroxy-vitamin D₃ or 1,25-dihydroxy-vitamin D₃ in a pharmaceutical composition, a health supplement or an animal feed.

27. The process according to any one of embodiments 1 to 12, 15 to 17 and 25, comprising oxidizing vitamin D₂ to 25-hydroxy-vitamin D₂ and formulating the 25-hydroxy-vitamin D₂ in a pharmaceutical composition, a health supplement or an animal feed.

28. The compound (1S,Z)-3-(2-((1R,3aS,7aR,E)-7a-methyl-1-((2R)-5-(2-methyloxiran-2-yl) pentan-2-yl) octahydro-4H-inden-4-ylidene)ethylidene)-4-methylenecyclohexan-1-ol (i.e. 25,26-epoxy-vitamin D₃).

29. The compound (3S,6R,E)-6-((1R,3aS,7aR,E)-4-((Z)-2-((S)-5-hydroxy-2-methylenecyclohexylidene)ethylidene)-7a-methyloctahydro-1H-inden-1-yl)-2-methylhept-4-ene-2,3-diol (i.e. 25,28-dihydroxy-vitamin D₂).

1. A process for oxidising a secosteroid, comprising the step of contacting said secosteroid with a mutant CYP102A (Cytochrome P450 family 102A sub-family member) enzyme, wherein said CYP102A enzyme comprises a heme monooxygenase domain comprising a P450 fold, and said mutant CYP102A enzyme comprises substitution(s) in the polypeptide chain of a wild-type CYP102A enzyme at one or more positions, thereby enhancing monooxygenase activity and/or altering product selectivity of the mutant enzyme.

2. The process according to claim 1, wherein said mutant CYP102A enzyme comprises:
(a) substitution(s) in the polypeptide chain of a wild-type CYP102A enzyme at one or more positions corresponding to amino acid residue positions 435, 82, 184, 260 and/or 72 of SEQ ID NO: 2; or
(b) one or more substitutions selected from E435I, E435M, E435T, A82M, A82L, A82I, A82F, A82W, A184I, T260G, T260A, S72A and/or S72G in SEQ ID NO:2, or corresponding substitutions in the polypeptide chain of a wild-type CYP102A enzyme at one or more positions corresponding to amino acid residues 435, 82, 184, 260 and/or 72 of SEQ ID NO: 2.

3. (canceled)

4. The process according to claim 2, wherein said mutant CYP102A enzyme additionally comprises:
(a) one or more substitutions at one or more positions corresponding to amino acid residue positions 29, 78, 87, 178, 263, 264, 268, 328 and/or 354 of SEQ ID NO:2; or
(b) one or more substitutions selected from L29M, V78F, F87A, F87V, F87I, F87S, F87T, V178F, V178W, V178L, V178I, I263G, A264G, T268S, A328G, and/or M354F or corresponding substitutions at one or more positions corresponding to amino acid residue positions 29, 78, 87, 178, 263, 264, 268, 328 and/or 354 of SEQ ID NO: 2.

5. (canceled)

6. The process according to claim 4, wherein said mutant CYP102A enzyme comprises a said substitution at position 82 of SEQ ID NO:2 or a position corresponding thereto and a said substitution at position 87 of SEQ ID NO:2 or a position corresponding thereto.

7. The process according to claim 6, wherein said mutant CYP102A enzyme comprises:
(a) a said substitution at position 82 of SEQ ID NO:2 or a position corresponding thereto and a said substitution at position 87 of SEQ ID NO:2 or a position corresponding thereto; and additionally comprises a said substitution at position 184 of SEQ ID NO:2 or a position corresponding thereto, a said substitution at position 260 of SEQ ID NO:2 or a position corresponding thereto, a said substitution at position 72 of SEQ ID NO:2 or a position corresponding thereto, and/or a said substitution at position 435 of SEQ ID NO:2 or a position corresponding thereto; or
(b) a said substitution at position 82 of SEQ ID NO:2 or a position corresponding thereto, a said substitution at position 87 of SEQ ID NO:2 or a position corresponding thereto and a said substitution at position 435 of SEQ ID NO:2 or a position corresponding thereto.

8. (canceled)

9. The process according to claim 1, wherein said mutant CYP102A enzyme comprises one of the following groups of substitutions in SEQ ID NO:2:
a. A82M/F87A;
b. A82M/T260G;
c. A82M/A184I;
d. A82M/S72A;
e. A82M/E435I, E435M or E435T;
f. F87A/T260G;
g F87A/A184I;
h. F87A/S72A;
i. A82M/F87A/T260G;
j. A82M/F87A/A184I;
k. A82M/F87A/E435I, E435M or E435T;
l. F87A/A184I/T260G;
m. F87A/A184I/S72A;
n. F87A/A184I/E435I, E435M or E435T;
o. F87A/A82M/A184I/T260G;
p. F87A/A82M/T260G/S72A;
q. F87A/A82M/A184I/T260G/S72A;
r. F87A, F87I or F87V/A82M/A184I/T260G/E435I, E435M or E435T;
S. F87A, F87I, F87V, F87S or F87T/A82M/A184I/T260G/S72A/E435I, E435M or E435T; or
t. F87I/A82M/A184I/T260G/S72A/E435M/L29M;
or corresponding substitutions in the polypeptide chain of a wild-type CYP102A enzyme at one or more positions corresponding to the amino acid residue positions of SEQ ID NO: 2 as listed in a.-t.

10. The process according to claim 1, wherein said mutant CYP102A enzyme additionally comprises one or more substitutions at one or more positions corresponding to amino acid residue positions 47, 51, 74, 171, 188, 239, 259, 307, 319, 330 and/or 353 of SEQ ID NO:2, optionally selected from R47L, Y51F, A74G, H171L, N239H, I259V, L188Q, Q307H, N319Y, A330V, A330I, A330L, A330W and/or L353I in SEQ ID NO:2, or corresponding substitutions in the polypeptide chain of a wild-type CYP102A enzyme at one or more positions corresponding to amino acid residue positions 47, 51, 74, 171, 188, 239, 259, 307, 319, 330 and/or 353 of SEQ ID NO: 2.

11. The process according to claim 1, wherein the mutant CYP102A enzyme comprises a fusion of a heme monooxygenase domain to a reductase domain.

12. The process according to claim 1, which is for:
(a) oxidizing vitamin D₃, optionally which is for producing 25-hydroxy-vitamin D₃, optionally further comprising oxidation of 25-hydroxy-vitamin D₃ to 1,25-dihydroxyvitamin D₃; or
(b) oxidizing vitamin D₂, optionally which is for producing 25-hydroxy-vitamin D₂.

13. (canceled)

14. The process according to claim 1, wherein the mutant CYP102A enzyme is a mutant CYP102A1 enzyme.

15. A mutant CYP102A (Cytochrome P450 family 102A sub-family member) enzyme, wherein said CYP102A enzyme comprises a heme monooxygenase domain comprising a P450 fold, and said mutant CYP102A enzyme comprises substitution(s) in the polypeptide chain of a wild-type CYP102A enzyme at one or more positions corresponding to amino acid residue positions 435, 82, 184, 260 and/or 72 of SEQ ID NO: 2, thereby enhancing monooxygenase activity and/or altering product selectivity of the mutant enzyme.

16. The mutant CYP102A enzyme according to claim 15, wherein said mutant CYP102A enzyme comprises one or more substitutions selected from E435I, E435M, E435T, A82M, A82L, A82I, A82F, A82W, A184I, T260G, T260A, S72A and/or S72G in SEQ ID NO:2.

17. A polynucleotide which comprises a sequence which encodes an enzyme as defined in claim 15, optionally in the form of a vector.

18. A cell which expresses an enzyme as defined in claim 15.

19. The cell according to claim 18 which is a prokaryotic or eukaryotic cell, optionally wherein the cell is a strain of Escherichia coli, Pseudomonas sp., yeast, Pichia sp., Rhodococcus sp., Bacillus sp.

20. A transgenic animal or plant whose cells are as defined in claim 18.

21. The process according to claim 1, wherein the secosteroid is oxidized in a cell which expresses said mutant CYP102A (Cytochrome P450 family 102A sub-family member) enzyme, wherein said mutant CYP102A enzyme comprises substitution(s) in the polypeptide chain of a wild-type CYP102A enzyme at one or more positions corresponding to amino acid residue positions 435, 82, 184, 260 and/or 72 of SEQ ID NO: 2, thereby enhancing monooxygenase activity and/or altering product selectivity of the mutant enzyme.

22. The process according to claim 1, comprising:
(a) oxidizing vitamin D₃ to 25-hydroxy-vitamin D₃ or 1,25-dihydroxy-vitamin D₃ and formulating the 25-hydroxy-vitamin D₃ or 1,25-dihydroxy-vitamin D₃ in a pharmaceutical composition, a health supplement or an animal feed; or
(b) oxidizing vitamin D₂ to 25-hydroxy-vitamin D₂ and formulating the 25-hydroxy-vitamin D₂ in a pharmaceutical composition, a health supplement or an animal feed.

23. (canceled)

24. A composition of matter comprising the compound (1S,Z)-3-(2-((1R,3aS,7aR,E)-7a-methyl-1-((2R)-5-(2-methyloxiran-2-yl) pentan-2-yl) octahydro-4H-inden-4-ylidene)ethylidene)-4-methylenecyclohexan-1-ol (i.e. 25,26-epoxy-vitamin D₃) or the compound (3S,6R,E)-6-((1R,3aS,7aR,E)-4-((Z)-2-((S)-5-hydroxy-2-methylenecyclohexylidene)ethylidene)-7a-methyloctahydro-1H-inden-1-yl)-2-methylhept-4-ene-2,3-diol (i.e. 25,28-dihydroxy-vitamin D₂).

25. (canceled)


SEQUENCE LISTING (36 sequences)

SEQ ID NO: 1 - DNA, 3144 bp, Bacillus megaterium
  source 1..3144: mol_type = genomic DNA
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaatgcgatgaatcacgctttgataaaaacttaagtcaagcggcacttaaatttgtacgtgattttgcaggagacgggttatttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgctccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgccaaaaaagcacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctggg

SEQ ID NO: 2 - protein, 1048 aa, Bacillus megaterium
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 3 - protein, 470 aa, Bacillus megaterium
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKV

SEQ ID NO: 4 - protein, 470 aa, Bacillus subtilis
KETSPIPQPKTFGPLGNLPLIDKDKPTLSLIKLAEEQGPIFQIHTPAGTTIVVSGHELVKEVCDEERFDKSIEGALEKVRAFSGDGLFTSWTHEPNWRKAHNILMPTFSQRAMKDYHEKMVDIAVQLIQKWARLNPNEAVDVPGDMTRLTLDTIGLCGFNYRFNSYYRETPHPFINSMVRALDEAMHQMQRLDVQDKLMVRTKRQFRYDIQTMFSLVDSIIAERRANGDQDEKDLLARMLNVEDPETGEKLDDENIRFQIITFLIAGHETTSGLLSFATYFLLKHPDKLKKAYEEVDRVLTDAAPTYKQVLELTYIRMILNESLRLWPTAPAFSLYPKEDTVIGGKFPITTNDRISVLIPQLHRDRDAWGKDAEEFRPERFEHQDQVPHHAYKPFGNGQRACIGMQFALHEATLVLGMILKYFTLIDHENYELDIKQTLTLKPGDFHISVQSRHQEAIHADVQAAEKAAP

SEQ ID NO: 5 - protein, 470 aa, Bacillus subtilis
KQASAIPQPKTYGPLKNLPHLEKEQLSQSLWRIADELGPIFRFDFPGVSSVFVSGHNLVAEVCDEKRFDKNLGKGLQKVREFGGDGLFTSWTHEPNWQKAHRILLPSFSQKAMKGYHSMMLDIATQLIQKWSRLNPNEEIDVADDMTRLTLDTIGLCGFNYRFNSFYRDSQHPFITSMLRALKEAMNQSKRLGLQDKMMVKTKLQFQKDIEVMNSLVDRMIAERKANPDENIKDLLSLMLYAKDPVTGETLDDENIRYQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQADIKA

SEQ ID NO: 6 - protein, 470 aa, Bacillus anthracis
DKKVSAIPQPKTYGPLGNLPLIDKDKPTLSFIKLAEEYGPIFRMQTLSDTIIVVSGHELVAEVCDETRFDKSIEGALAKVRAFAGDGLFTSETQEPNWQKAHNILMPTFSQRAMKDYHAMMVDIAVQLVQKWARLNPNENVDVPEDMTRLTLDTIGLCGFNYRFNSFYRETPHPFITSMTRALDEAMHQLQRLDIEDKLMWRTKRQFQHDIQSMFSLVDNIIAERKSSENQEENDLLSRMLNVQDPETGEKLDDENIRFQIITFLIAGHETTSGLLSFAIYFLLKNPDKLKKAYEEVDRVLTDSTPTYQQVMKLKYIRMILNESLRLWPTAPAFSLYAKEDTVIGGKYPIKKGEDRISVLIPQLHRDKDAWGDNVEEFQPERFEELDKVPHHAYKPFGNGQRACIGMQFALHEATLVMGMLLQHFEFIDYEEYQLDVKQTLTLKPGDFKIRIVPRNQTISHTTVLAPTEE

SEQ ID NO: 7 - protein, 470 aa, Bacillus cereus
EKKVSAIPQPKTYGPLGNLPLIDKDKPTLSFIKIAEEYGPIFQIQTLSDTIIVVSGHELVAEVCDETRFDKSIEGALAKVRAFAGDGLFTSETHEPNWKKAHNILMPTFSQRAMKDYHAMMVDIAVQLVQKWARLNPNENVDVPEDMTRLTLDTIGLCGFNYRFNSFYRETPHPFITSMTRALDEAMHQLQRLDIEDKLMWRTKRQFQHDIQSMFSLVDNIIAERKSSGDQEENDLLSRMLNVPDPETGEKLDDENIRFQIITFLIAGHETTSGLLSFAIYFLLKNPDKLKKAYEEVDRVLTDPTPTYQQVMKLKYMRMILNESLRLWPTAPAFSLYAKEDTVIGGKYPIKKGEDRISVLIPQLHRDKDAWGDNVEEFQPERFEELDKVPHHAYKPFGNGQRACIGMQFALHEATLVMGMLLQHFELIDYQNYQLDVKQTLTLKPGDFKIRILPRKQTISHPTVLAPTED

SEQ ID NO: 8 - protein, 470 aa, Bradyrhizobium japonicum
SSKNRLDPIPQPPTKPVVGNMLSLDSAAPVQHLTRLAKELGPIFWLDMMGSPIVVVSGHDLVDELSDEKRFDKTVRGALRRVRAVGGDGLFTADTREPNWSKAHNILLQPFGNRAMQSYHPSMVDIAEQLVQKWERLNADDEIDVVHDMTALTLDTIGLCGFDYRFNSFYRRDYHPFVESLVRSLETIMMTRGLPFEQIWMQKRRKTLAEDVAFMNKMVDEIIAERRKSAEGIDDKKDMLAAMMTGVDRSTGEQLDDVNIRYQINTFLIAGHETTSGLLSYTLYALLKHPDILKKAYDEVDRVFGPDVNAKPTYQQVTQLTYITQILKEALRLWPPAPAYGISPLADETIGGGKYKLRKGTFITILVTALHRDPSVWGPNPDAFDPENFSREAEAKRPINAWKPFGNGQRACIGRGFAMHEAALALGMILQRFKLIDHQRYQMHLKETLTMKPEGFKIKVRPRADRERGA

SEQ ID NO: 9 - protein, 470 aa, Bacillus licheniformis
NKLDGIPIPKTYGPLGNLPLLDKNRVSQSLWKIADEMGPIFQFKFADAIGVFVSSHELVKEVSEESRFDKNMGKGLLKVREFSGDGLFTSWTEEPNWRKAHNILLPSFSQKAMKGYHPMMQDIAVQLIQKWSRLNQDESIDVPDDMTRLTLDTIGLCGFNYRFNSFYREGQHPFIESMVRGLSEAMRQTKRFPLQDKLMIQTKRRFNSDVESMFSLVDRIIADRKQAESESGNDLLSLMLHAKDPETGEKLDDENIRYQIITFLIAGHETTSGLLSFAIYLLLKHPDKLKKAYEEADRVLTDPVPSYKQVQQLKYIRMILNESIRLWPTAPAFSLYAKEETVIGGKYLIPKGQSVTVLIPKLHRDQSVWGEDAEAFRPERFEQMDSIPAHAYKPFGNGQRACIGMQFALHEATLVLGMILQYFDLEDHANYQLKIKESLTLKPDGFTIRVRPRKKEAMTAMPGAQPEENG

SEQ ID NO: 10 - protein, 470 aa, Bacillus thuringiensis
DKKVSAIPQPKTYGPLGNLPLIDKDKPTLSFIKLAEEYGPIFQIQTLSDTIIVVSGHELVAEVCDETRFDKSIEGALAKVRAFAGDGLFTSETDEPNWKKAHNILMPTFSQRAMKDYHAMMVDIAVQLVQKWARLNPNENVDVPEDMTRLTLDTIGLCGFNYRFNSFYRETPHPFITSMTRALDEAMHQLQRLDIEDKLMWRTKRQFQHDIQSMFSLVDNIIAERKSSENQEENDLLSRMLNVQDPETGEKLDDENIRFQIITFLIAGHETTSGLLSFAIYFLLKNPDKLKKAYEEVDRVLTDSTPTYQQVMKLKYIRMILNESLRLWPTAPAFSLYAKEDTVIGGKYPIKKGEDRISVLIPQLHRDKDAWGDDVEEFQPERFEELDKVPHHAYKPFGNGQRACIGMQFALHEATLVMGMLLQHFEFIDYEDYQLDVKQTLTLKPGDFKIRIVPRNQTISHTTVLAPTEE

SEQ ID NO: 11 - protein, 470 aa, Bacillus weihenstephanensis
DKKVSAIPQPKTYGLLGNLPLIDKDKPTLSFIKIAEEYGPIFRIQTLSDTIIVVSGHELVAEVCDETRFDKSIEGALAKVRAFAGDGLFTSETHEPNWKKAHNILMPTFSQRAMKDYHAMMVDIAVQLVQKWARLNPNENVDVPEDMTRLTLDTIGLCGFNYRFNSYYRETPHPFITSMSRALDEAMHQLQRLDIEDKLMWRTKRQFQHDIQSMFSLVDNIIAERKSSGNQEENDLLSRMLNVQDPETGEKLDDENIRFQIITFLIAGHETTSGLLSFAIYFLLKNPDKLKKAYEEVDRVLTDPTPTYQQVMKLKYIRMILNESLRLWPTAPAFSLYAKEDTVIGGKYPIKKGEDRISVLIPQLHRDKDAWGDNVEEFQPERFEELDKIPHHAYKPFGNGQRACIGMQFALHEATLVMGMLLQHFEFIDYQDYQLDVKQTLTLKPGDFKIRILPRNQTISHTTVLAPIEE

SEQ ID NO: 12 - protein, 470 aa, Erythrobacter litoralis
DAPTALAPIPQPPGKPIVGNAFTVDSSRLIQSLMELAEEYGPIFQLEVMGTPLVFVSGADMVAEICDESRFDKTVRGPLKRLRLIAGDGLFTGDTDDPNWAKAHHILLPSFSQKAMGSYLPMMTDIASQLMLKWERLNSDDVIDVPMDMVRLTLDTIGVCGFGYRFNSFYREDFHPFIEALNRTLDTTQKMRGLPGEKLLKRQQIEQLNEDAAYMNNLVDEIIRERRQTGESGQGDLLDFMLSGRDPVTGERLSDENIRYQINTFLIAGHETTSGLLSFTLYYLLKNRDVLQRAYAEVDEVLGRNIDQTPTLSQIGRLPYIRAILSEALRLWPTAPAMGLAPFEDEVLGGKYAIAKGTFTTVLIPSLHRDKLVWGENPEAFNPDNFSPKAEAARPPHAYKPFGNGQRACIGRQFAIQESILVLGMLLQRFELFDHADYQLRIKETLSIKPDGFTIKARLRHDVERGGVAT

SEQ ID NO: 13 - protein, 470 aa, Erythrobacter sp.
ATNATLTPIPQPPGKPLIGNALTVDASQQIQSLMELAEEYGPIFQLDMMGTPIVIISGADLVAEVCDEKRFDKSVRGPLKRLRLIGGDGLFTGDTDAPNWSKAHNILLPSFSQKAMGSYLPMMTDIATQLVMKWERMNSDDVIDVPKDMIRLTLDTIGVCGFGYRFNSFYREDFHPFIRALTRTLETTQKIRGIPGEKLLKGDAVKQLHRDAKYMNNLVDEIIRERQRSGGDGPEDLLDFMLSGRDPLTGERLSDENIRYQINTFLIAGHETTSGLLSFTLYYLLKNRDVLTRAYAEVDTVLGRNIDQPPSLKQIGQLPYIRAILFEALRLWPTAPAFGLAPFEDEVLGGKYLIPKGTFTTVLIPSLHRDKSVWGENPEVFDPENFTAEAEAARPPHAYKPFGNGQRACIGRQFAIQESILVLAMILQRFELFDHSDYKLDIKETLSIKPDEFTIKARMRKDVERGGKAT

SEQ ID NO: 14 - protein, 470 aa, Rhodopseudomonas palustris
SSSNKLAPIPHPPKQPVVGNMLSIDTKAPVQHLVRLAEELGPIFWLDMMGAPIVIVSGYDLVDEISDEKRFDKAVRGALRRVRTVGGDGLFTADTSEPNWSKAHNILLTPFGGRAMQSYHPSMVDIAEQLVKKWERLNADDEIDVVHDMTALTLDTIGLCGFDYRFNSFYRRDYHPFVESLVRSLETIMMTRGLPLENLWMKKRRDTLAEDVAFMNAMVDEIIAERRKAAAVADKMDMLGAMMTGVDKVTGEPLDDVNIRYQINTFLIAGHETTSGLLSCAIYALLKHPEVLQKAYDEVDRVLGADTSVEPSYQQVNQLGYITQILKETLRLWPPAPAYGVAPIQDETIGGQYHLKRGTFTTVLVLALHRDPSIWGPNPDAFDPENFSREAESKRPANAWKPFGNGQRACIGRGFAMHEAALALGMILQRFKLIDHTRYRMVLKETLTIKPEGFKIKVRPRSDKDRATRI

SEQ ID NO: 15 - protein, 470 aa, Rhodopseudomonas palustris
ASNNKMSPIPQPPTRPVVGNMLSLDSAAPVQDLTRLAKELGPIFWLDMMGAPIVIVSGYTLVDELSVETRLDKVVRGALRRVRAIGGDGLFTADTAEPNWSKARNILLQPFGNRAMQSYHPSMVDIAEQLVKKWERLNADDEIDVVHDMTALTLDTIGLCGFDYRFNSFYRRDYHPFVESLVRSLETIMMIRGLPLENFWMRRRRSDLATDVAFMNKMVDEIVAERRKSAEASDGKKDMLNAMMSGVDRSTGEQLDDVNIRYQINTFLIAGHETTSGLLSYAIYALLKHPDVLKKAYAEVDRVLGADIEARPSYQQVTQLTYITQILKEALRLWPPAPAYGIAPLKDETIAGGKYSLKKNTFISILVTALHHDPAVWGPNPDLFDPENFSPEAEAKRPVNAWRPFGNGQRACIGRGFAMHEAALALGMILQRFKLIDHQRYQIRLKETLTIKPDGFKIKVRPRSGHDRTV

SEQ ID NO: 16 - protein, 470 aa, unidentified; identical to SEQ ID NO: 15

SEQ ID NO: 17 - protein, 470 aa, Bacillus sp.
QQTSIIPKPKTYGPFKNIPHIKKGELSQTFWRLADELGPIFQFEFSKATSIFVSNHELFQEICDESRFDKYIGTSLNKVRAFAGDGLFTSWTEEPNWRKAHHILMPAFSQQAMKGYHEMMLDIATQLVQKWQRTGRDEEIEVAEDMTKLTLDTIGLCGFDFRFNSFYKENQHPFIESMVNGLSEAMDQASRLPVADKLMIKRRKKFEENVDFMKQLVDDIIQERKKQDKTGDDLLSLMLHAKDPETGERLSDENIRYQIITFLIAGHETTSGLLSFAIYFLLKNPEKLKKAVQEADDVLQGGLPTFKQVQKLNYTRMVLNESLRLWPTAPTFSLYAKEDTVIGGKYSIEKNQSVSVLLPKLHRDQAVWGEDAEEFKPERFLHPEKIPQHAYKPFGNGQRACIGMQFALHEATMVLAMVLHNLELIDHTSYELDLKESLTIKPNDFKIKVRPRKQQLFMVPPKEETKKSTT

SEQ ID NO: 18 - protein, 470 aa, Bacillus sp.
DKKVSAIPQPKTYGPLGNLPLIDKDKPTLSFIKLAEEYGPIFQIQTLSDTIIVVSGHELVAEVCDETRFDKSIEGALAKVRAFAGDGLFTSETHEPNWKKAHNILMPTFSQRAMKDYHAMMVDIAVQLVQKWARLNPNENVDVPGDMTRLTLDTIGLCGFNYRFNSFYRETSHPFITSMTRALDEAMHQLQRLDIEDKLMWRTKRQFQHDIQSMFSLVDNIIAERKSSGNQEENDLLSRMLHVQDPETGEKLDDENIRFQIITFLIAGHETTSGLLSFAIYFLLKNPDKLKKAYEEVDRVLTDPTPTYQQVMKLKYIRMILNESLRLWPTAPAFSLYAKEDTVIGGKYPIKKGEDRISVLIPQLHRDKDAWGDNVEEFQPERFEELDKVPHHAYKPFGNGQRACIGMQFALHEATLVMGMLLQHFEFIDYEDYKLDVKQTLTLKPGDFKIRIVPRNQTISHTTVLAPTEE

SEQ ID NO: 19 - protein, 470 aa, Bacillus marmarensis
AAKQESKKHDDKHKGQIPGPKGLPIIGSLLDIDLTDSLKSIIDMAKDYPKLFALNIGGNTEVMICSRELMDELSDESRFHKLVVGGVEKLRPLAGDGLFSAQHNNQEWAIAHRILMPLFGPLTIREMFPDMRDISEQLCLKWARLGSSSTIDVGNDFTRLTLDTIALCTMGFRFNSFYSNDKMHPFVESMVAALIDAEKQSFLPDVVQSLRIRAQSHFKKHAAVMKSTCQDILDQRRKNPVEGKDLLNAMMNGKDPKTGMGMSDGNIVDNLITFLIAGHETTSGLLSFAFYYLLENPEKLQRAREEVDEVLGDENITADHLPKMPYINMIFRETLRLMPTAPGFYVTPFKDEVIGGQYNVSAGDPLFLFLHMIHRDPEVWGPDAEEFRPERMADEHFNKLPKNAWKPFGNGMRGCIGREFAWQEAQIVTIMLLQNFDMVKADPNYKLKIKQSLTIKPDGFNMKVKLREGR

SEQ ID NO: 20 - protein, 470 aa, Pontibacillus halophilus
TQTHRTIPQAKSYGPLGSLPLIDREKPIQSFLKLADVYGPIYQFQYPGKQSTFVSGYNLVKELSDESRFDKKVGPALQKVRAFGGDGLFTSETNEANWKKAHNILLPSFSQQAMKGYHDKMTDLAVQLVQKWARLNPNETADVPEDMTRLTLDTIGLCGFNYRFNSFYRETNHWFVDSMVRALDESMNQLQRLGIQDKLMVKAKRQFDQDIHDMFSLVDQLIAERKAAGDQGEDDLLAHMLKGKDPETGEQLSDENIRFQIITFLIAGHETTSGMLSFAIYYLMKNPDKLQKAYEEVDEVFGDQGVPSYKQVKQLKYVRNVLNESLRLWPTAPAFALYAKDDTTLGDDYHLEKGQTAVILTPALHRDQSVWGDDVESFKPERFENPQDIPQHAFKPFGNGQRACIGQQFAMHEATLVLGMLLQNFEFIDHADYELEIKESLTLKPDGMTIQVKQRKPLNLTRATDEHNAE

SEQ ID NO: 21 - protein, 470 aa, Ktedonobacter racemifer
LRSQLDTIPMGDMKEVHPESAVRDLIKQAQRHGPIFQLPLPDGTRRIVLSSFALVDEVCNDQFFDKRVSVNVRNLRSAIGSGLFSADTSDPNWRKAHTILMPSFSLQAMRNYEPMMVDAAEQLMAKWEHLNPEDLINVPDDMTRVTLETIGLCGFSYHFHAITREQMHPFVEAMVHILSTTAARATRPPIEEKLRFRQRRQLQADLDLLLSTADQLVRERKMMGTEGQKYNDLLNAMLNGIDKQTGEKLDDVNIRNQIITFLIAGHETTSGLLSFATYFLLHHPDVLNRAYEEVDRVLGRDTRKTPTYEQIHQLVYIKQILKETLRLCPTAPMFNRYPYEERPLAGKYQLQPSDTITVLTPMLHRDRSIWGDDAEIFNPDHHFNPEAEKQRPANAYKPFGTGQRSCIGREFALQEATLVLGMMLQRFYPYTTGIYQLKIREALTIKPADFYMRVRPRTDIDHIIITPLDE

SEQ ID NO: 22 - protein, 470 aa, Ktedonobacter racemifer
QIEEKTLVSIPQPQKKPLVGNLYSLDHALPTLSMMDLARELGPIYQLEIPGRRTVIVSGFELADELSDETRFDKRVWRPLQNVRAFAGDGLFTAYTQEANWHKAHNILLPNFSMRAMQGYIPMMQDIAEQLLGKWERMNANEEIDVVSDMTRLTLDTIGLCGFDYRFNSFYREDLHPFIQSMGNALGESMARGRRLALQEKLMVNKHRQFQADVDFMNNLVDQVIRERKAGGENAQKKDLLSYMLSGVDKQSGEGLNDVNIRYQIITFLIAGHETTSGLLSFSLYFLLKHPEVLAKAYDEVDRVLGPDTSAKPTFQQINKLQYVSQILKESLRLWPTAPLFSVYPYEETVVGDKYRMDKDSEWAILIPMLHRDKSVWGEDADEFNPDHFSHEAEASRPANAYKPFGNGERACIGRQFAMQEATLVLGQILQRFNLLDPHNYELKIKQTLTLKPEGFTMKVRKRSDAERNL

SEQ ID NO: 23 - protein, 470 aa, Streptomyces coelicolor
AQTAREPARDGLPKGFRSAELGWPELHRIPHPPYRLPLLGDVVGASRRTPMQDSLRYARRLGPIFRRRAFGKEFVFVWGAALAADLADEARFAKHVGLGVANLRPVAGDGLFTAYNHEPNWQLAHDVLAPGFSREAMAGYHVMMLDVAARLTGHWDLAEASGRAVDVPGDMTKLTLETIARTGFGHDFGSFERSRLHPFVTAMVGTLGYAQRLNTVPAPLAPWLLRDASRRNAADIAHLNRTVDDLVRERRANGGTGGGTGSGSGSGDLLDRMLETAHPRTGERLSPQNVRRQVITFLVAGHETTSGALSFALHYLAQHPDVAARARAEVDRVWGDTEAPGYEQVAKLRYVRRVLDESLRLWPTAPGFAREAREDTVLGGTHPMRRGAWALVLTGMLHRDPEVWGADAERFDPDRFDAKAVRSRAPHTFKPFGTGARACIGRQFALHEATLVLGLLLRRYELRPEPGYRL

SEQ ID NO: 24 - protein, 21 aa, synthetic construct
CDESRFDKNLSQALKFVRDFA

SEQ ID NO: 25 - protein, 21 aa, synthetic construct
DKNLSQALKFVRDFAGDGLFT

SEQ ID NO: 26 - protein, 21 aa, synthetic construct
SQALKFVRDFAGDGLFTSWTH

SEQ ID NO: 27 - protein, 21 aa, synthetic construct
FVRDFAGDGLFTSWTHEKNWK

SEQ ID NO: 28 - protein, 21 aa, synthetic construct
DQPHPFITSMVRALDEAMNKL

SEQ ID NO: 29 - protein, 21 aa, synthetic construct
ITSMVRALDEAMNKLQRANPD

SEQ ID NO: 30 - protein, 21 aa, synthetic construct
DDENIRYQIITFLIAGHETTS

SEQ ID NO: 31 - protein, 21 aa, synthetic construct
NIRYQIITFLIAGHETTSGLL

SEQ ID NO: 32 - protein, 21 aa, synthetic construct
IRYQIITFLIAGHETTSGLLS

SEQ ID NO: 33 - protein, 21 aa, synthetic construct
IITFLIAGHETTSGLLSFALY

SEQ ID NO: 34 - protein, 21 aa, synthetic construct
LNEALRLWPTAPAFSLYAKED

SEQ ID NO: 35 - protein, 21 aa, synthetic construct
EYPLEKGDELMVLIPQLHRDK

SEQ ID NO: 36 - protein, 21 aa, synthetic construct
DHTNYELDIKETLTLKPEGFV